(19) United States
(12) Patent Application Publication
(10) Pub. No.: US 2024/0207302 A1
(43) Pub. Date: Jun. 27, 2024

(54) ANTIVIRAL COMPOUNDS, METHODS FOR THE MANUFACTURING OF COMPOUNDS, ANTIVIRAL PHARMACEUTICAL COMPOSITION, USE OF THE COMPOUNDS AND METHOD FOR THE ORAL TREATMENT OF CORONAVIRUS INFECTION AND RELATED DISEASES THEREOF

(71) Applicants: João Batista CALIXTO, Florianópolis-SC (BR); Jaime Alberto RABI NALLAR, Rio de Janeiro - RJ (BR); Thiago Moreno LOPES E SOUZA, Rio de Janeiro - RJ (BR)

(72) Inventors: João Batista CALIXTO, Florianópolis-SC (BR); Jaime Alberto RABI NALLAR, Rio de Janeiro - RJ (BR); Thiago Moreno LOPES E SOUZA, Rio de Janeiro - RJ (BR)

(21) Appl. No.: 18/553,666

(22) PCT Filed: Apr. 1, 2022
(86) PCT No.: PCT/BR2022/050120

§ 371 Date: Oct. 2, 2023

(30) Foreign Application Priority Data
Apr. 1, 2021 (WO) PCT/BR2021/050136

(51) Int. Cl. A61K 31/7076; A61K 31/4025; A61K 31/4178; A61K 31/4184; A61K 31/513; A61K 31/52; A61K 31/5365; A61P 31/14; C07D 473/34; C07H 19/167

(52) U.S. Cl. CPC A61K 31/7076; A61K 31/4025; A61K 31/4178; A61K 31/4184; A61K 31/513; A61K 31/52; A61K 31/5365; A61P 31/14; C07D 473/34; C07H 19/167

(57) ABSTRACT

The present invention relates to antiviral compounds selected from cytokinins, their nucleosides and nucleotide analogs, and their prodrugs as inhibitors of viral RNA synthesis, or their salts, solvates, derivatives, or even combinations of aforementioned compounds, for prophylactic treatment, curative (therapeutic) or mitigative coronavirus infection, represented by human and veterinary coronavirus, SARS-COV-2 and MHIV, and for the treatment of individuals potentially exposed to COVID-19. The present invention also comprises the methods for the manufacturing of such compounds, the antiviral pharmaceutical composition containing the compounds of the invention, as well as the use of the compounds, combinations of compounds, and method for the prophylactic, curative (therapeutic) or mitigative treatment of coronavirus infection, represented by coronavirus, in especial SARS-COV-2 and of patients with COVID-19, individual infected with SARS-COV-2 or potentially exposed to this virus. The antiviral activity of the compounds of this invention against SARS-COV-2 was greatly enhanced by inhibiting the 3′-5′-exonuclease. Synergistic results of the compounds according to the present invention were obtained from the combination with repurposed drugs.

### FIELD OF THE INVENTION

The present invention comprises antiviral compounds encompassing purine bases, their nucleosides, nucleotides and related manufacturing methods to impair the viral RNA synthesis in members of the coronavirus family aiming at the prevention, treatment and cure of individuals with 2019 coronavirus disease (COVID-19). The antiviral pharmaceutical composition containing the compounds of the invention, as well as the use of compounds, combinations of compounds and compositions, and method for the use of compounds in COVID-19 are claimed henceforward. In certain embodiments, the disclosure relates to certain purines, nucleosides and nucleotides prodrugs, monophosphate and diphosphates and active triphosphates or salts thereof comprising the class of cytokinins, such as zeatin (MB-907), zeatin riboside (MB-804), kinetin (MB-905), kinctin riboside (MB-801), their nucleotides and phosphoramidates prodrugs (MB-711), particularly kinetin riboside 5′-triphosphate (MB-717).

### BACKGROUND OF THE INVENTION

Pathogenic coronaviruses are a major threat to global public health, as exemplified by severe acute respiratory syndrome coronavirus (SARS-COV), Middle East respiratory syndrome coronavirus (MERS-COV), and the newly emerged SARS-COV-2, the causative agent of 2019 coronavirus disease (COVID-19). Genetic analysis of SARS-COV-2 revealed its proximity to SARS-like beta corona viruses of bat origin, bat-SL-CoVZC45 and bat-SL-CoVZXC21 (1). It is believed that throughout the year of 2021, SARS-COV-2 provoked the death of some 18 million people worldwide. Besides the highly pathogenic coronaviruses (CoV), other members of the Coronaviridae family, like the viral species 229E, NL63, HKUI and OC43 provoke seasonal infections in humans. Members of this family possess positive viral RNA that are transcribed and replicated within the host-cell. All the members of this family share from 70 to 100% homology in the machinery to replicate the viral genome.

Until recently, the most effective and used response to control the SARS-COV-2 pandemic was social distancing, as an attempt to avoid contact between infected and uninfected individuals and flatten the virus dissemination curve. While social actions can disrupt virus transmission rates, they are not expected to reduce the absolute number of infected individuals. Furthermore, these strategies are also provoking a severe reduction in global economic activity.

Several vaccines have already been approved and other are under development. However, SARS-COV-2 has mutated into more contagious mutants that are challenging the efficiency of the available vaccines. These mutations, are so far, concentrated in the spike proteins of the virus. Additionally, three direct acting small molecule antivirals agents passed the stage of clinical tests. The injectable Remdesivir (Gilead Sciences) and the orally available Molnupiravir (Merck) received emergence authorization by FDA. Remdesivir and Molnupiravir were designed to inhibit the RNA synthesis. Valoxavir (Pfizer), an inhibitor of virus major protease (Mpro) also received an authorization of use before approval.

Even in countries where vaccination against Covid-19 pandemic it is beyond 70% and herd immunity is expected, new variants continuously emerge and threat the public health system.

The clinic manifestation of COVID-19 ranges from influenza-like illness to severe systemic complication, leading to death. Disease progression to severity may occur within days or weeks overlapping with SARS-COV-2 migration from upper to lower respiratory tract. Either resident cells of the respiratory tract or others migrating to this system are susceptible of infection as long as they possess the receptor for viral entry the: angiotensin-converting enzyme 2 (ACE2). As judged by autopsy from postmortem samples of COVID-19 patients, it is known that type II pneumocytes in the lungs succumb to SARS-COV-2 infection, limiting their ability to produce type C surfactant to maintain pulmonary compliance. In the course of the natural history of the infection, respiratory impairment, and intense viral production in the acute phase of severe COVID-19, progress to uncontrolled pro-inflammatory disease associated with leukopenia and coagulopathy in critically ill patients. The general proinflammatory state alone can increase vascular permeability and death of endothelial cells, which can generate a positive feed-back for the migration of cells from the immune system to the lungs, with the consequent death of these cells and leukopenia in the peripheral blood. Coagulation disorders occur as a consequence of viral-induced cell death, exposing pro-coagulant signals, such as Von Willebrand and Tissue Factor (CD142), and recruiting platelets. The interaction of platelets with monocytes and other cells also exacerbates inflammation. Therefore, SARS-COV-2 actively replicates mainly in type II pneumocytes, leading in some individuals to cytokine storm and the exacerbation of thrombotic pathways. Besides the virus-triggered pneumonia and sepsis-like discase associated with severe COVID-19, SARS-COV-2 may reach the central nervous system and liver. Early blockage of the natural clinical evolution of infection by direct acting antivirals will be likely able to prevent the disease progression to severe COVID-19. Indeed, clinical trials providing early antiviral intervention accelerated the decline of viral loads and slowed disease progression. The decrease of viral loads is expected to be a critical laboratory parameter, because lowering viral shedding may protect the individual and reduce transmissibility thus benefiting the population as a wholc.

To effectively address the worldwide burden caused by SARS-COV-2 on infected individuals, and society as a whole, it is essential to identify new antiviral drugs for immediate use (repurposing), as well as to develop new, more effective, and selective chemical entities and vaccines for medium to long-term solutions to prevent and treat the clinical spectrum of SARS-COV-2 infection.

In recent years bioinformatics has been used as a technique to suggest potential drugs for some pathologies. However, drug discovery remains a scientific challenge that requires intensive scientific empirical investigation.

Recent scientific publications and patent references based on computational drug discovery or in silico data already suggested the use of well-known antiviral drugs to treat COVID-19. These included dolutegravir, raltegravir, daclatasvir, ombitasvir and pibrentasvir. Albeit without experimental evidences. Computational approaches to predict active candidates against SARS-COV-2 have suggested, without reduction to practice, that kinetin riboside or zeatin riboside could possess antiviral properties.

The World Health Organization (WHO) proposed an emergency strategy to combat COVID-19 pandemic attempting to repurpose known drugs. Lopinavir (LPV)/ritonavir (RTV), combined or not with interferon-β (IFN-β), chloroquine (CQ) and hydroxychloroquine (HCQ) and remdesivir (RDV) were initially investigated under the auspicious of the Solidarity trial. Lack of unequivocal clinical benefit paused the enthusiasm for CQ, HCQ and LPV/RTV. In line with natural history of infection, RDV showed promising results in non-human primates and in a limited number of clinical studies as long as it was provided carly after the onset of illness. Because of initial positive results with RDV against COVID-19, this drug received an emergency authorization by the Food and Drugs Administration (FDA). Despite that, global access to RDV is limited because of its price resulting in part for the difficulties in the manufacturing procedure. In addition, RDV has limited oral bioavailability and is subjected to marked liver extraction where it is preferentially converted into its active form. RDV is an adenosine-like monophosphoramidate pro-drug that needs to be converted in its triphosphate to induce a late termination of coronavirus RNA synthesis. Another nucleoside analog, N-4-hydroxycytidine-5′-isopropyl ester, EIDD-2801 or MK-4482, is orally bioavailable and has been showed to present antiviral activity against coronaviruses including SARS-, MERS-, and SARS-COV-2. MK-4482 is a prodrug of the nucleotide triphosphate of N4-hydroxycytidine (NHC), which exerts its antiviral action through introduction of an error-prone viral RNA replication, after its incorporation in the viral genome. Although MK-4482 was tested in a preliminary human study for “Safety, Tolerability, and Pharmacokinetics” in healthy volunteers in the UK and US, there are certain concerns because of the observed in vitro toxicity, including for human cells. Nevertheless, in the context of the emergency response against COVID-19, this drug was moved forward into efficacy clinical trials for treatment for COVID-19 and received FDA's authorization for limited use.

Favipiravir is a pyrazine analog with broad activity against RNA viruses. This pro-drug is up-taken by the salvage pathway and converted to its riboside monophosphate first. Despite initial controversial results, suggesting very low potency against SARS-CoV-2 and restricted clinical studies with COVID-19, infected animals ameliorated with high doses of favipiravir. Thus, several clinical studies, with dosages above 1.5 g/day are ongoing against COVID-19.

AT-527, a C6-aminomethyl guanosine analog, is the hemi-sulfate salt of AT-511, a novel phosphoramidate prodrug of 2′-fluoro-2′-C-methylguanosine-5′-monophosphate being developed by ATEA pharmaceuticals. This prodrug, which is orally bioavailable, presented an in vitro potency against SARS-COV-2-infected hepatic cell in the micromolar range. It recently failed to reach the expected end point and new clinical studies are underway.

RDV, favipiravir, molnupiravir, and AT-527 are prodrugs of their corresponding triphosphates that are incorporated in the nascent viral RNA by the RNA-dependent RNA polymerase. These drugs also target the orthologue enzyme in SARS-COV-2 replication cycle, also known as non-structural protein 12 (nsp12). Moreover, to conduct transcription and replication, SARS-COV-2 nsp12 associates with other viral non-structural proteins in a coordinated catalytic complex. This unique replicase/transcription complex carries out the synchronized activity of other nonstructural proteins: a viral helicase (nsp13); the holo-RNA polymerase (its co-factors nsp7 and 8, and the main RNA-dependent RNA polymerase enzyme, the nsp12; the 3′,5′-exonuclease (nsp13), the endonuclease (nsp15) and the methyltransferases (nsp14 and nsp16). This multi-step event presents several opportunities to inhibit the viral replication. Since the enzymatic machinery in coronaviruses is highly conserved, SARS-COV-2 may be considered as a prototypic species for the development of antiviral compounds for the Coronaviridae family as a whole.

For all the above, including the ability of SARS-COV-2 variants to escape the immune response, the limitations of the recently approved antiviral drugs and the failure of most if not all repurposing efforts, the necessity of new, more effective, and specific drugs continues to be an urgent objective.

Over the years pyrimidines, purines, their nucleoside and nucleotide analogs have proven to be a rich source of antiviral agents. RDV, MK-4482, and AT-527 seem to reconfirm the great anti Covid-19 potential of this family of compounds. Thus, it is relevant to further explore the potential of this source in search for orally available and potent direct acting antiviral therapies.

### SUMMARY OF THE INVENTION

The present invention provides compounds, pharmaceutical compositions and methods/uses for treating and/or preventing SARS-COV-2 viral infection that were selected from purines, their nucleoside and nucleotide analogs capable to inhibit coronavirus, in especial SARS-COV-2 RNA synthesis. Also included are their derivatives, salts, solvates or prodrugs, or even combinations of compounds, for the prophylactic treatment, post-exposure (therapeutic) treatment of COVID-19 and for the treatment of individuals potentially exposed to or at risk of exposure to coronaviruses.

Other embodiments of the present invention include the pharmaceutical composition, comprising: (i) the effective antiviral amounts of one or more compounds of the invention, their derivatives, salts, solvates or prodrugs, or even combinations of the abovementioned compounds, for the prophylactic, curative or mitigative treatment of SARS-COV-2 infection and for the treatment of individuals with COVID-19; and (ii) pharmacologically acceptable excipient(s) compatible with the active ingredients.

In addition, the present invention relates to uses of the compounds and compositions of the invention for the manufacture of an antiviral drug to: (i) inhibit the SARS-CoV-2 RNA synthesis; and (ii) for prophylactic, curative or mitigative treatment for SARS-COV-2 infection and for the treatment of individuals with COVID-19.

An embodiment of the present invention is also the method for the prophylactic, curative or mitigative treatment of SARS-COV-2 infection, of an individual infected with SARS-COV-2 or potentially exposed to SARS-COV-2, where it is treated with a therapeutically effective amount of one or more antiviral compounds of the invention. Furthermore, a lead compound is no genotoxic and safe (no toxic), according to acute and 28 days repeated toxicology and safe to cardiovascular system according to hERG and telemetry assay.

### BRIEF DESCRIPTION OF THE FIGURES

FIG. 1A, 1B and 1C. The antiviral activity of the compounds against SARS-CoV-2. Vero (A), HuH-7 (B) and Calu-3 (C) cells, at density of 5×10⁴ cells/well in 96-well plates, were infected with SARS-COV-2, for 1 h at 37° C. Inoculum was removed, cells were washed and incubated with fresh Dulbecco's modified cagle medium, DMEM, containing 2% fetal bovine serum (FBS) and the indicated concentrations of the compounds. Vero (A) cells were infected with MOI of 0.01 and cell-monolayers were lysed after 24 h. HuH-7 (B) cells were infected with MOI of 0.1 and cell-monolayers were lysed after 48 h. Calu-3 (C) cells were infected with MOI of 0.5 and cell-monolayers were lysed after 48-72 h. Total RNA was extracted, viral RNA synthesis was quantified by detection of sub-genomic RNA at region of the gene N by real time RT-PCR. The data represent means±SEM of three independent experiments performed with three technical replicates per experiment. The asterisks indicate P values below 0.05.

FIG. 2A and 2B. The antiviral activity of the compounds against SARS-COV-2 production of infectious virus particles. Calu-3 cells (human type II pneumocytes), at density of 5×10⁴ cells/well in 96-well plates, were infected with SARS-COV-2, at MOI of 0.5 for 1 h at 37° C. Inoculum was removed, cells were washed and incubated with fresh DMEM containing 2% fetal bovine serum (FBS) and the indicated concentrations of the compounds were added just in this moment (A) or also in the following days (B). After 48-72 h, cell supernatants were harvested and infectious viral titers in the culture supernatant were measured by PFU/mL in Vero cells. MB-905, its corresponding ribonucleoside (MB-801) and monophosphoramidate (MB-711) are displayed. Remdesivir (RDV) and MK-4482 were used as positive controls. The data represent means±SEM of at least three independent experiments performed with three technical replicates per experiment.

FIG. 3A and 3B. The anti-coronavirus activity of compound MB-905 requires the engagement of the enzyme adenine phosphoribosyl transferase (APRT). (A) HuH-7 cells, at density of 5×10⁴ cells/well in 96-well plates, were infected with SARS-CoV-2, at MOI of 0.1 for 1 h at 37° C., treated with indicated concentrations of MB-905, in the presence or absence of 10 μM of adenine, or with its 9-tetrahydopyranyl derivative (MB-906). After 48 h, cell-monolayers were lysed, total RNA extracted and viral RNA synthesis was quantified by detection of sub-genomic RNA at region of the gene N by real time RT-PCR. (B) Calu-3 cells (human type II pneumocytes), at density of 5×10⁴ cells/well in 96-well plates, were infected with SARS-COV-2, at MOI of 0.5 for 1 h at 37° C. treated with indicated concentrations of MB-905, in the presence or absence of 10 μM of adenine, or with MB-906. After 48-72h, cell supernatants were harvested and infectious viral titers in the culture supernatant were measured by PFU/mL in Vero cells (B). The data represent means±SEM of at least three independent experiments performed with three technical replicates per experiment.

FIG. 4. Reduction of SARS-COV-2 associated RNA synthesis in type II pneumocytes. Calu-3 cells (5×10⁵ cells/well in 48-well plates) were infected with SARS-COV-2 at MOI of 0.5, for 1 h at 37° C. Inoculum was removed, cells were washed and incubated with fresh DMEM containing 2% fetal bovine serum (FBS) and the indicated compounds were added at 10 μM. After 48 h-72 h, cells monolayers were lysed, total RNA extracted, and quantitative RT-PCR performed for detection of ORF1 and ORFE mRNA. The data represent means±SEM of three independent experiments. * P<0.05 for comparisons with vehicle (DMSO). #P<0.05 for differences in genomic and sub-genomic RNA. The data represent means±SEM of three independent experiments performed with three technical replicates per experiment.

FIG. 5A, 5B and 5C. The compounds impair SARS-COV-2 RNA synthesis and SARS-COV-2-induced release of inflammatory mediators in human primary monocytes. Human primary monocytes were infected at the MOI of 0.01 and treated with indicated concentrations of the compounds. After 24 h, cell-associated virus RNA loads (A), as well as TNF-α (B) and IL-6 (C) levels in the culture supernatant were measured. The data represent means±SEM of experiments with cells from at least three healthy donors. Differences with P<0.05 are indicates (*), when compared to untreated cells (nil) to each specific treatment. The data represent means±SEM of three independent experiments performed with three technical replicates per experiment.

FIG. 6A and 6B. The antiviral activity of the compounds against SARS-COV-2 production of infectious virus particles is enhanced by co-inhibition by exonuclease. Calu-3 cells (human type II pneumocytes), at density of 5×10⁴ cells/well in 96-well plates, were infected with SARS-COV-2, at MOI of 0.5 for 1 h at 37° C. Inoculum was removed, cells were washed and incubated with fresh DMEM containing 2% fetal bovine serum (FBS) and the indicated concentrations of the compounds were added. After 48 -72 h, cell supernatants were harvested and infectious viral titers in the culture supernatant were measured by PFU/mL in Vero cells. MB-905, MB-801, MB-711 and MB-804 were used at 10 μM. Remdesivir (RDV), sofosbuvir and tenofovir were used as positive controls at a concentration of 10 μM. Inhibition of viral exonuclease was achieved by HIV integrase inhibitors raltegravir (A) or dolutegravir (B) at 5 μM. The data represent means±SEM of at least three independent experiments performed with three technical replicates per experiment. * Indicate P<0.05 statistical difference comparing to infected and untreated cell (nil). #Indicate P<0.05 statistical difference comparing a specific drug as monotreatment vs its use as co-treatment with raltegravir (A) or dolutegravir (B).

FIG. 7A and 7B. MB-905 induces transitions and transversion in the SARS-CoV-2 genome. Huh-7 cells at density of 2×10⁶ cells were infected at MOI of 0.1 for 1 h at 37° C. and treated with MB-905 at 0.5 μM, initially. Cells were monitored daily up to the observation of cytophatic effects (CPE). Virus was recovered from the culture supernatant, tittered and used in a next round of infection in the presence of higher drug concentration. These passages occurred for three months period and covered the MB-905 concentrations from 0.5 to 9 μM. As a control, SARS-COV-2 was also passaged in the absence of treatments to monitor genetic drifts associated with culture. At each passage, total RNA was extracted from culture supernatant, by Qiamp viral RNA, and 4.2 ng was used for libraries construction using the MGIEasy RNA Library Prep Set. All libraries were constructed through RNA-fragmentation (250 bp), followed by reverse-transcription and second-strand synthesis. After purification with MGIEasy DNA Clean Beads, libraries were quantified and loaded onto the flow cells (MGI-2000). Mega 7.0 software was used for alignment and base statistics. Samples were run in quadruplicates. Only sequences with quality score phread above Q36 were considered. Average coverage was above 10.000-fold. (A) The evolutionary history of the sequencing passages was inferred by using the Maximum Likelihood method and Kimura-2 parameter model, with 1000 boostraps. The phylogenetic tree is rooted by Wuhan-01 index case (#EPI_ISL_402125), MB-905-associated sequences are in red and control sequences (nil) are in green. (B) Base use statistics use in relation to the codon position, comparing changes in the MB-905-treated sequences over the untreated control. As a proxy of cDNA sequencing, positions assigned as T are equivalent to U in the original RNA. Two- and 1.5-fold change is statistically significant at P<0.01 and P<0.05, respectively. Sequences are deposited on GISAID, under accession code #EPI_ISL_1023783, EPI_ISL_1023784, EPI_ISL_1023786, EPI_ISL_1023788, EPI_ISL_1023790, EPI_ISL_1023792, EPI_ISL_1023794, EPI_ISL_1023796, EPI_ISL_1023798, EPI_ISL_1023800, EPI_ISL_1023801, EPI_ISL_1023803, EPI_ISL_1023805, EPI_ISL_1023807, EPI_ISL_1023809, EPI_ISL_1023811, EPI_ISL_1023812, EPI_ISL_1023815, EPI_ISL_1023816, EPI_ISL_1023818, EPI_ISL_1023820, EPI_ISL_1023822, EPI_ISL_1023824, EPI_ISL_1023826, EPI_ISL_1023827, EPI_ISL_1023829, EPI_ISL_1023831, EPI_ISL_1023833, EPI_ISL_1023835, EPI_ISL_1023837, EPI_ISL_1023839, EPI_ISL_1023841, EPI_ISL_1023843, EPI_ISL_1023845.

FIG. 8A to 8F. Oral and intravenous pharmacokinetic for MB-905 in both mice ([FIG. 8A]-C) and rats ([FIG. 8D]-F). The number of animals is shown in each figure legends. The vertical lines represent the mean±the standard error deviation.

FIG. 9A to 9D. MB-905 increases survival of Swiss mice infected by the prototypic beta-coronavirus murine hepatitis virus (MHV). Three to six-month old Swiss Webster outbreed mice were infected by intranasal inoculation of 3×10⁴ PFU of MHV and treated daily by oral gavage with 250 mg/kg/day of MB-905, since the second day after infection. As a control, daclatsvir (DAC) was used to inhibit the beta-coronavirus replication, at 60 mg/kg/day, starting also on the second day after infection. (A) comparison of SARS-COV-2 and MHV main enzymes involved with virus replication, the RNA-dependent RNA polymerase (nsp12, YP_009924352.1 vs YP_009725307.1) and exonuclease (nsp14, YP_009924354.1 vs YP_009725309.1). (B) Kaplan-Meier survival curve of MHV-infected animals untreated (nil; black; n=18), treated with MB-905 (green; n=10) or DAC (red; n=10). (C) Evolution of percentual weight change upon MHV infection in comparison to mock-infected (uninfected) control. (D) Total cell counts, as a proxy of cellular inflammation, in the bronchoalveolar lavage of the animals at the eleventh day after infection. * Indicate P<0.05 compared to nil-treated mice.

FIG. 10A and 10B. Effect of MB-905 on body weight in repeated dose 28-day oral toxicity study in mice. Body weight gain in weeks (A) in males and (B) in females by 4 weeks. Animals were treated daily with vehicle (10 mL/Kg, p.o.), MB-905 (10, 80 or 250 mg/kg, p.o.). Body weight was measured once a week. Data are presented as mean±S.E.M. Statistical analysis was performed by two-way ANOVA followed by Bonferroni post hoc test. n=10 animals per group.

FIG. 11A to 11F. Male Sprague-Dawley rats (9-12 weeks), previously submitted to surgery for placement of the DSI™ PhysioTel hardware system implant in the abdominal aorta, were treated orally with Vehicle (5 ml/kg) or MB-905 (50 or 250 mg/kg) once a day for 7 consecutive days. Cardiovascular parameters such as systolic blood pressure (A), diastolic blood pressure (B), mean blood pressure (C), hert rate (D), body temperature (E) and body temperature change (F) were evaluated before treatments (bascline) and at 0.5, 1, 2, 3, 4, 5, 6, 7, 12 and 24 hours after first treatment with MB-905. Data are presented as mean±S.E.M. Statistical analysis was performed by two-way ANOVA followed by Bonferroni post hoc test. n=4-6 animals per group.

FIG. 12A to 12D. Male Sprague-Dawley rats (9-12 weeks), previously submitted to surgery for placement of the DSI™ PhysioTel hardware system implant in the abdominal aorta, were treated orally with Vehicle (5 ml/kg) or MB-905 (50 or 250 mg/kg) once a day for 7 consecutive days. Cardiovascular parameters such as QT interval (A), QTc interval (B), QRS interval (C) and PR internval (D) were evaluated before treatments (baseline) and at 0.5, 1, 2, 3, 4, 5, 6, 7, 12 and 24 hours after first treatment with MB-905. Data are presented as mean±S.E.M. Statistical analysis was performed by two-way ANOVA followed by Bonferroni post hoc test. n=4-6 animals per group.

FIG. 13A to 13F. Male Sprague-Dawley rats (9-12 weeks), previously submitted to surgery for placement of the DSI™ PhysioTel hardware system implant in the abdominal aorta, were treated orally with Vehicle (5 ml/kg) or MB-905 (50 or 250 mg/kg) once a day for 7 consecutive days. Cardiovascular parameters such as systolic blood pressure (A), diastolic blood pressure (B), mean blood pressure (C), hert rate (D), body temperature (E) and body temperature change (F) were evaluated before treatments (baseline) and at 0.5, 1, 2, 3, 4, 5, 6, 7, 12 and 24 hours after 7 days of treatment with MB-905. Data are presented as mean±S.E.M. Statistical analysis was performed by two-way ANOVA followed by Bonferroni post hoc test. n=4-6 animals per group.

FIG. 14A to 14D. Male Sprague-Dawley rats (9-12 weeks), previously submitted to surgery for placement of the DSI™ PhysioTel hardware system implant in the abdominal aorta, were treated orally with Vehicle (5 ml/kg) or MB-905 (50 or 250 mg/kg) once a day for 7 consecutive days. Cardiovascular parameters such as QT interval (A), QTc interval (B), QRS interval (C) and PR internval (D) were evaluated before treatments (baseline) and at 0.5, 1, 2, 3, 4, 5, 6, 7, 12 and 24 hours after 7 days of treatment with MB-905. Data are presented as mean±S.E.M. Statistical analysis was performed by two-way ANOVA followed by Bonferroni post hoc test. n=4-6 animals per group.

FIG. 15A and 15B. Inhibition of voltage-dependent potassium channels of the hERG type (human ether-a-go-go related). HEK293 cell line (4×10e5 cell) (BPS Bioscience, San Diego, CA, USA) expressing recombinant human ERG potassium channel (ether-a-gogo-related gene, Kv11.1) was used. The channel activity was determined using FLIPR Potassium assay kit (Molecular Devices—San Jose, CA, USA). Cells were cultivated in microplate and incubated with a loading buffer for one hour at room temperature in the dark. Then, MB-905 (0.01-300 μM) or Dofetilide (0.0001-1 μM, used as positive control drug) were added to the wells and incubated for thirty minutes at room temperature in the dark. After that, the microplate was transferred to FlexStation 3 (Molecular Devices—San Jose, CA, USA) with the addition of 1 mM thallium+10 mM potassium using automated pipetting. Data analysis was performed using SoftMax Pro Software (Molecular Devices—San Jose, CA, USA) and GraphPad Prism. The results were expressed as percentage of inhibition of the hERG channel and the mean inhibitory concentration (IC50) was determined. Concentration-response curve for MB-905 and an inhibitor (Dofetilide) on the hERG channel by Potassium Assay Kit. HEK293 cells transfected with hERG were incubated with MB-905 (0.01-300 μM; or with the Reference compound (0.0001-1 μM; Dofetilide) for 30 minutes. Then, the addition of 1 mM Thallium+10 mM Potassium was carried out through the automatic pipetting present in the FlexStation 3 equipment. MB-905); (B) Relative inhibition of the hERG channel after incubation of positive control drug Dofetilide. Data analyzes were performed using GraphPad Prism. The results were expressed as percentage of inhibition of the hERG channel and the inhibitory concentration (IC50) was performed through non-linear regression of the data generated from the fluorescence intensity values. The data in the graph were expressed as mean±standard error of the mean of three experiments independent. The vertical bars represent the mean of 3 independent experiments.

FIG. 16A to 16F. MB-905 inhibits SARS-COV-2 RNA synthesis. SARS-COV-2 RNA polymerase (nsp12, nsp7 and nsp8, BPSBiosciences #100839) were incubated with a 33-mer template, 10-mer primer, NTPs and MgC12 for 3 h at 37° C. As nucleotide incorporation into the newly synthesized strand releases pyrophosphate, this product was further quantified by commercial luminescent assay (Lonza Bioscience, LT07-610). MB905-ribose (801) triphosphate (801-TP) was assayed, along with GS-443902 (equivalent to remdesivir triphosphate), as a positive control (A). Calu-3 cells (5×10⁵ cells/well in 48-well plates) were infected with SARS-COV-2 at MOI of 0.5, for 1 h at 37° C. Inoculum was removed, cells were washed and incubated with fresh DMEM containing 2% fetal bovine serum (FBS) and the indicated compounds were added at 10 μM. After 48 h-72 h, cells monolayers were lysed, total RNA extracted, and quantitative RT-PCR performed for detection of ORF1 and ORFN mRNA (B). For panel B, *P<0.05, ** P<0.01, *** P<0.001, when compared with vehicle (DMSO). Cell-associated viral RNA, from mock- and SARS-COV-2-infected calu-3 cells treated or not with MB-905, was incubated with anti-kinctin antibody (Ab), or unspecific IgG (in isotype control; Iso), coupled with protein A conjugated to magnetic beads. After washing, ORFN mRNA and cellular housekeeping genes (GAPDH) were quantified by real time RT-PCR (C). For panel C, *P<0.05 and #P<0.05, for comparisons of specific groups. SARS-COV-2-infected calu-3 cells treated or not with MB-905 were monitored daily up to the observation of cytophatic effects (CPE). Virus was recovered from the culture supernatant, titered and used in a next round of infection in the presence of higher drug concentration. These passages occurred for three months period and covered the MB-905 concentrations from 0.5 to 9 μM. As a control, SARS-COV-2 was also passaged in the absence of treatments to monitor genetic drifts associated with culture. At each passage, total RNA was extracted from culture supernatant, libraries constructed using the MGIEasy RNA Library Prep Set and sequenced (MGI-2000). Mega 7.0 software was used for alignment and base statistics. Samples were run in quadruplicates. Only sequences with quality score phread above Q36 were considered. Average coverage was above 10,000-fold. Base use statistics use in relation to the codon position, comparing changes in the MB-905-treated sequences over the untreated control is scored with statistical significance if *P<0.05 (D). As a proxy of cDNA sequencing, positions assigned as T are equivalent to U in the original RNA (D). Infected calu-3 cells were treated with the indicated concentrations of the MBs with raltegravir (E) or dolutegravir (F), followed by titration in Vero cells. For panels E and F, *P<0.05 for statistical difference comparing to monotreatment vs combination and #P<0.05 for statistical difference comparing treatments vs nil-treated cells. The data represent means±SEM of three independent experiments.

FIG. 17A to 17E. The antiviral and anti-inflammatory activities of the MBs against SARS-COV-2. Calu-3 cells (human type II pneumocytes), at density of 5×10⁴ cells/well in 96-well plates, were infected with SARS-COV-2, at MOI of 0.1 for 1 h at 37° C. Inoculum was removed, cells were washed and incubated with fresh DMEM containing 2% fetal bovine serum (FBS) and the indicated concentrations of the compounds were added just in this moment (A) or also in the following days (B). After 48 -72 h, cell supernatants were harvested and infectious viral titers in the culture supernatant were measured by PFU/mL in Vero cells. The data represent means±SEM of at least three independent experiments on calu-3 cells followed by titration with technical duplicates in vero cells. Human primary monocytes were infected at the MOI of 0.1 and treated with indicated concentrations of the compounds. After 24 h, cell-associated virus RNA loads (C), as well as IL-6 (D) and TNF-α (E) levels in the culture supernatant were measured. The data represent means±SEM of experiments with cells from at least three healthy donors of monocytes. MB-905, its corresponding ribonucleoside (MB-801) and monophosphoramidate (MB-711) are displayed. Remdesivir (RDV) and molnupiravir (MK-4482) were used as positive controls. Differences with P<0.05 are indicates (*), when compared to untreated cells (nil) to cach specific treatment.

FIG. 18. MB-905 induces transitions and transversion in the SARS-COV-2 genome. Huh-7 cells at density of 2×10⁶ cells were infected at MOI of 0.1 for 1 h at 37° C.and treated with MB-905 at 0.5 μM, initially. Cells were monitored daily up to the observation of cytophatic effects (CPE). Virus was recovered from the culture supernatant, tittered and used in a next round of infection in the presence of higher drug concentration. These passages occurred for three months period and covered the MB-905 concentrations from 0.5 to 9 μM. As a control, SARS-COV-2 was also passaged in the absence of treatments to monitor genetic drifts associated with culture. At cach passage, total RNA was extracted from culture supernatant, by Qiamp viral RNA, and 4.2 ng was used for libraries construction using the MGIEasy RNA Library Prep Sct. All libraries were constructed through RNA-fragmentation (250 bp), followed by reverse-transcription and second-strand synthesis. After purification with MGIEasy DNA Clean Beads, libraries were quantified and loaded onto the flow cells (MGI-2000). Mega 7.0 software was used for alignment and base statistics. Samples were run in quadruplicates. Only sequences with quality score phread above Q36 were considered. Average coverage was above 10,000-fold. (A) The evolutionary history of the sequencing passages was inferred by using the Maximum Likelihood method and Kimura-2 parameter model, with 1,000 boostraps. The phylogenetic tree is rooted by Wuhan-01 index case (#EPI_ISL_402125), MB-905-associated sequences are in red and control sequences (nil) are in green. Sequences are deposited on GISAID, under accession code #EPI_ISL_1023783, EPI_ISL_1023784, EPI_ISL_1023786, EPI_ISL_1023788, EPI_ISL_1023790, EPI_ISL_1023792, EPI_ISL_1023794, EPI_ISL_1023796, EPI_ISL_1023798, EPI_ISL_1023800, EPI_ISL_1023801, EPI_ISL_1023803, EPI_ISL_1023805, EPI_ISL_1023807, EPI_ISL_1023809, EPI_ISL_1023811, EPI_ISL_1023812, EPI_ISL_1023815, EPI_ISL_1023816, EPI_ISL_1023818, EPI_ISL_1023820, EPI_ISL_1023822, EPI_ISL_1023824, EPI_ISL_1023826, EPI_ISL_1023827, EPI_ISL_1023829, EPI_ISL_1023831, EPI_ISL_1023833, EPI_ISL_1023835, EPI_ISL_1023837, EPI_ISL_1023839, EPI_ISL_1023841, EPI_ISL_1023843, EPI_ISL_1023845.

FIG. 19A and 19B. Molecular model of promiscuous pairing of tautomeric kinctin. A) Three conformations in which furfuryl from kinetin affects double-strand conformation with neighbor A. This model is in line with a possible steric hindrance of the RNA polymerase. B) Three conformations in which furfuryl from kinetin affects double-strand conformation with neighbor C and G. This model is in line with error-prone mechanism.

FIG. 20A to 20K. MB905 is antiviral, anti-inflammatory and survival of transgenic K18 mice infected with SARS-COV-2 gamma-infected. Transgenic mice expressing hACE2 receptor to SARS-COV-2 entry at age of 10-12 weeks old were infected with 10⁵ PFU intranasally. After 12 -18 h the treatments were performed and maintained daily. A) 7-day survival summary of infected and treated animals. B) Change in body weight. C) clinical score variation. The groups treated with MB905 at 140 mg/kg/day, alone or in combination with dolutegravir (DTG), and their controls, were analyzed for viral RNA levels (D), titers (E) and LDH activity (F) in the bronchoalveolar lavage (BAL). These groups were analyzed by H&E staining of lung parenchyma (G). ELISA assays to quantify TNF (H), IL-6 (I), KC (J) and cell counts (K) were also performed in the BAL. * P<0.05. Number of animals per group varied from 8 to 20 distributed in three independent experiments, data represent mean±SEM.

FIG. 21. Pharmacokinetics of MB-905 in three pre-formulations in mice. A) Single oral doses (3, 30 and 300 mg/kg) pharmacokinetics properties of MB-905 (5% tween 80+95% polyethylene glycol 400) in mouse plasma (32 2); B) Single oral doses (3, 30 and 300 mg/kg) pharmacokinetics properties of MB-905 (5% carboxymethylcellulose) in mouse plasma (n=1-2); c, Single oral doses (3, 30 and 550 mg/kg) pharmacokinetics properties of MB-905 (5% ethanol, 30% propylene Glycol, 45% polyethylene glycol 400 and 20% water) in mouse plasma (n=2-6); Data were expressed as mean±standard error of the mean.

### DETAILED DESCRIPTION OF THE INVENTION

The present invention relates to antiviral compounds endowed with ability to inhibit coronavirus, in especial SARS-COV-2, RNA synthesis, or their derivatives, salts, solvates or prodrugs, or even combinations of aforementioned compounds, in especial in combination with raltegravir and dolutegravir, for prophylactic treatment, cure or mitigation of coronavirus, in especial SARS-COV-2, infection and for the treatment of individuals potentially exposed or at risk of COVID-19.

In the state of the art, it is accepted that the term “analog” preferably refers to compounds in which one or more atoms or groups of atoms have been replaced by one or more atoms or groups of different atoms. Thus, the terms “nitrogenous bases, nucleoside and nucleotide analogs” refer to nitrogenous bases, nucleoside and nucleotide analogs in which one or more atoms or groups of atoms have been replaced by one or more atoms or groups of atoms other than those normally found in nucleosides/nucleotides.

In the state of the art, it is accepted that the terms “derivative” or “variant” refer preferentially to compounds that are derived from similar ones through chemical reactions, or to compounds that originate from a similar starting compound.

In the present application, the terms “analog,” “derivatives” or “variants” are included, as noted above.

The terms “nitrogenous bases, nucleoside and nucleotide analogs,” as used in the present application, refer to purines, their nucleosides and/or nucleotides, as well as the conversion or derivation from one form to another, found in an isolated or simultancous manner.

The term “viral RNA synthesis,” as used in the present application, refers to machinery to synthetize de novo viral RNA, which may require following SARS-CoV-2 non-structural proteins (nsp): helicase (nsp13), RNA polymerase (composed of the co-factors nsp7 and 8, and the main RNA-dependent RNA polymerase enzyme the nsp12), the exonuclease (nsp14/10), endonuclease (nsp15) and the methyltransferases (nsp10/14 and nsp16/10).

It is well known that there is an urgent need for drugs to treat SARS-COV-2 infection. Although molnupiravir and valoxavir are orally available, they still raise concerns on their applicability. As explored above, molnupiravir renders NHC inside the cells, which has mutagenicity potential to the host. Valoxavir has a quick metabolism, requiring the concomitant use of cytochrome P450 blockers, such as ritonavir. Thus, valoxavir/ritonavir have very broad drug interaction with different compounds, including those used in the supportive and palliative treatment of COVID-19 patients. Recently, great efforts have been made to understand the biology of this new disease, as well as to establish experimental models in vitro for the research and selection of potential viral targets and effective drugs.

The inventive antiviral activity described here for more than one member of the coronavirus family, SARS-COV-2 and MHV, makes the presumption that other coronaviruses of biomedical and veterinary interest are susceptible to the compounds and combinations; such as, canine coronavirus, feline coronavirus, human coronavirus 229E, porcine epidemic diarrhea virus, transmissible gastroenteritis virus, bovine coronavirus, canine respiratory coronavirus, human coronavirus OC43, human coronavirus NL63, human coronavirus HKU1, porcine hemagglutinating encephalomyelitis virus, puffinosis virus, rat coronavirus, turkey coronavirus, avian infectious bronchitis virus, avian infectious laryngotracheitis virus, SARS-COV, MERS-CoV, bovine respiratory coronavirus, human enteric coronavirus 4408, enteric coronavirus, equine coronavirus, and unclassified coronavirus.

Components of nucleic acids, amino bases and nucleosides exhibit antimicrobial activity, including antiviral. However, the practice of using knowledge already existing in the state of the art requires attention and care, specially the one obtained from a preliminary computational work, as the treatment of a disease is not only limited to the genetic information of the pathogen, but also to the information related to the pathogen-host relationship. This premise becomes more striking in a viral infection, because the pathogen depends almost strictly on the host's cellular system.

The present invention reveals that SARS-COV-2 RNA synthesis is inhibited in different cellular models (Vero African green monkey kidney cells, Huh-7 human hepatoma cells, calu-3 human type II pneumocytes, and in human primary monocytes) by the compounds disclosed in this invention. The compounds consistently inhibited the production of infectious virus particles in calu-3 human type II pneumocytes. Levels of inflammatory mediators were decreased by the compounds. Inhibition by MB-905 is synergized by exonuclease/endonuclease inhibitors. MB-905 impairs SARS-COV-2 codon usage and enhanced survival of infected mice by MHV.

In particular, this invention discloses nitrogenous bases, nucleoside and nucleotide analogs antiviral compounds that inhibit viral RNA synthesis are useful for the treatment, prevention and mitigation of SARS-COV-2 infection and for the treatment of potentially infected patients or individuals at risk of COVID-19.

The structure, chemical formula, and molecular weight of the antiviral compounds of the present invention are listed in table below. Such data is enough to clearly identify the compounds. The identification MB plus number is only used in this text to facilitate the reading, but it can clearly understand by the person skilled in art based on the data included table below.

TABLE 1
Identification of the structure of the compounds used in the present invention.

| compound | nomenclature | structure |
|---|---|---|
| MB-711 | kinetin riboside monophosphoramidate | 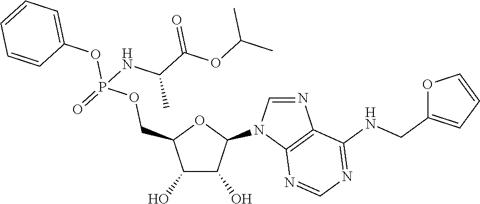 [SMILES: CC(C)OC(=O)[C@H](C)NP(=O)(OC[C@H]1O[C@@H](n2cnc3c(NCc4ccco4)ncnc32)[C@H](O)[C@@H]1O)Oc1ccccc1] |
| | | Chemical Formula: C₂₇H₃₃N₆O₉P |
| | | Molecular Weight: 618.5678 |
| MB-717 | kinetin-ribose 5'-triphosphate | 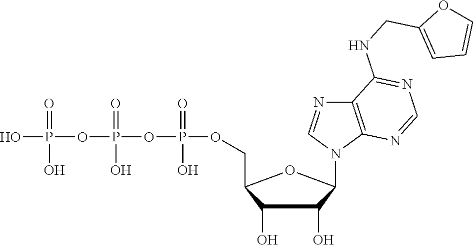 |
| | | Chemical Fornuis: C₁₅H₂₀N₅O₁₄P₈ |
| | | Molecular Weight: 587.27 |
| MB-801 | kinetin riboside | 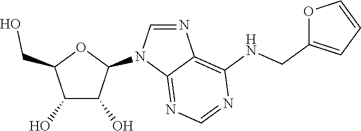 [SMILES: OC[C@H]1O[C@@H](n2cnc3c(NCc4ccco4)ncnc32)[C@H](O)[C@@H]1O] |
| | | Chemical Formula: C₁₈H₁₇N₅O₈ |
| | | Molecular Weight: 347.3310 |
| MB-804 | zeatin riboside | 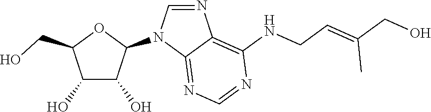 [SMILES: C/C(=C\CNc1ncnc2c1ncn2[C@@H]1O[C@H](CO)[C@@H](O)[C@H]1O)CO] |
| | | Chemical Formula: C₁₅H₂₁N₆O₆ |
| | | Molecular Weight: 351.3630 |
| MB-905 | kinetin | 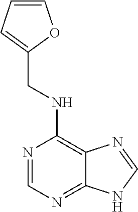 [SMILES: c1coc(CNc2ncnc3[nH]cnc23)c1] |
| | | Chemical Formula: C₁₀H₉N₆O |
| | | Molecular Weight: 215.2160 |
| MB-907 | zeatin | 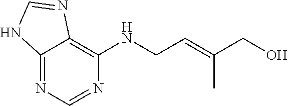 [SMILES: C/C(=C\CNc1ncnc2[nH]cnc12)CO] |
| | | Chemical Formula: C₁₀H₁₈N₅O |
| | | Molecular Weight: 219.2480 |

The compounds of table 1 are included in the scope of the present invention, along with their active monophosphate, diphosphates and triphosphates derivatives or salts thereof, preferably the triphosphates derivatives, when applicable.

In a preferred embodiment, the present invention refers to the compound kinetinribose 5′-triphosphate (MB-717). It was surprisingly discovered that phosphate derivatives, particularly triphosphate form, are able to inhibit viral RNA polymerase as a nucleotide analogue and that incorporation into viral RNA requires the formation of the nucleotide triphosphate.

The results presented in the examples below show cytokinins being incorporated into the viral nucleic acid. This incorporation into viral RNA requires the formation of the nucleotide triphosphate, as verified from in vivo lung tissue monitoring. Therefore, this is the metabolic pathway of the compounds according to the present invention.

General methods for providing the compounds of the present invention are well known in the art or described in the chemical literature, using the methods described herein or by combination thereof.

General synthetic schemes for preparing representatives compounds of the present invention are described below. These schemes are illustrative and are not meant to limit the possible techniques one skilled in the art may use to prepare the compounds disclosed herein.

Different methods to prepare the compounds of the present invention will be evident to those skilled in the art. Additionally, the various steps in the synthesis may be performed in an alternate sequence in order to give the desired compound or compounds.

The following abbreviations are used in the synthetic schemes detailed herein:
DCM: Dichloromethane
Py: Pyridine
THF: tetrahydrofuran
EtOH: Ethanol
MeOH: Methanol
Et₃N: triethylamine
DIAD: Diisopropyl azodicarboxylate
PPh₃: Triphenylphosphine
TrCl: Triphenylmethyl chloride
LiAlH₄: Lithium aluminium hydride
H₄N₂.H₂O: Hydrazine hydrate
PTSA: p-Toluenesulfonic acid
Me₂(OMe)₂: 2,2-dimethoxypropane
CSA: Camphorsulfonic acid
(CH₂OH)₂: Ethylene glycol

The compounds can be prepared, for example, by coupling 6-chloropurines or 6-chloropurine ribosides with appropriate aryl or alkyl amines in the presence of suitable tertiary base, as triethylamine, in alcoholic solvents such as ethanol or iso-propanol under reflux conditions, as shown in scheme 1.

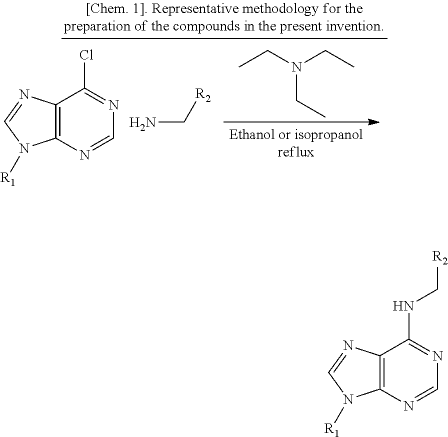

Methodologies for the preparation of the compounds according to the present invention is exemplified in the attached examples. Therefore, in a second embodiment, the present invention also refers to specific preparation of compounds according to the present invention.

The ability of nitrogenous bases, nucleoside and nucleotide analogs inhibitors of viral RNA synthesis to inhibit viral replication can be demonstrated by any assay capable of measuring or demonstrating decreased viral RNA load or infectious virus titers over cell cultures.

Although the prior art theoretically predicted through bioinformatics analysis that kinetin-ribose could be used as an antiviral, the person skilled in the art would not insist in the research as the preliminary results show that such a compound was able of modestly reducing the expression of the incoming SARS-COV-2 receptor on host cells, ACE2. It was surprisingly verified that kinetin-ribose or zeatin-ribose as prodrugs could not enough act on the RNA polymerase of SARS-COV-2 for it is a nucleoside, i.e., a prodrug that would need to have been converted to its phosphate, particularly triphosphate form, to then inhibit viral RNA polymerase as a nucleotide analogue.

Invention also features pharmaceutical compositions containing (i) an effective amount of one or more antiviral compounds of nitrogenous bases, nucleoside and nucleotide analogs inhibitors, or their salts, solvates, derivatives or prodrugs of such compounds according to the present invention, and (ii) pharmaceutically acceptable excipient (s) and compatible with the active ingredient, for the prophylactic, curative or mitigative treatment of coronavirus, in especial SARS-COV-2, infection and for the treatment of patients with or individuals at risk of COVID-19, and (iii) the combination of the compounds described here with inhibitors of the viral exonuclease/endonuclease, such as raltegravir, dolutegravir or their analogs.

More specifically, the present invention relates to the pharmaceutical composition having cytokinins, including kinetin, kinetin riboside, kinetin monophosphoramidate, zeatin and zeatin riboside, as well as active monophosphate and diphosphates and triphosphates thereof, particularly kinctin-ribose 5′-triphosphate (MB 717), as antiviral compounds for inhibiting coronavirus, in especial SARS-COV-2, viral replication, alone and in combination with raltegravir and dolutegravir or their analogs.

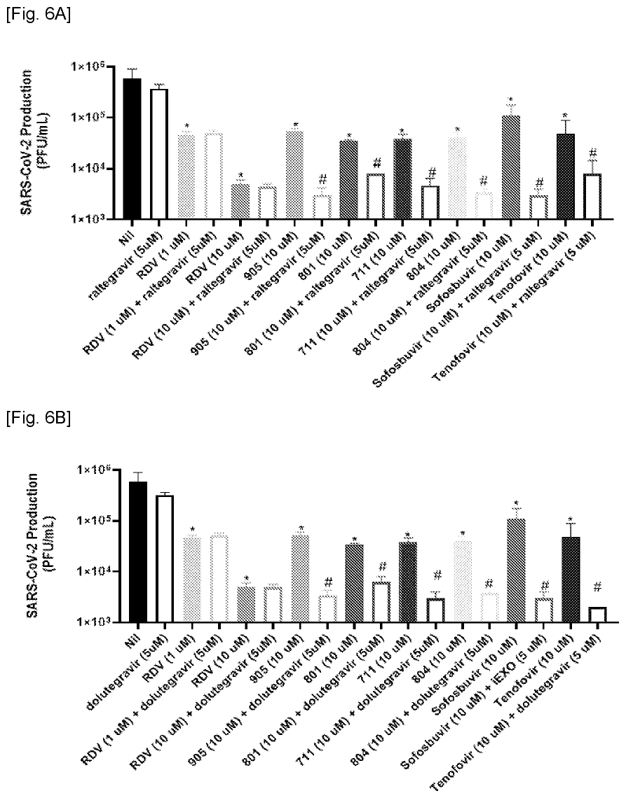

In an alternative embodiment, the present invention also refers to specific combinations with (i) sofosbuvir or tenofovir or their analogs and/or (ii) raltegravir, dolutegravir, pibrentasvir, ombitasvir and dacltasvir or their analogs. These specific combinations showed remarkable profile for the prophylactic, curative or mitigative treatment of coronavirus, in especial SARS-COV-2, infection and for the treatment of patients with or individuals at risk of COVID-19 as shown in FIGS. 6A and 6B attached herein.

The composition according to the present invention can comprise from 1 to 3,000 mg of the antiviral compounds, preferably from 1 to 500 mg. More preferably 10 mg, 15 mg, 25 mg, 30 mg, 40 mg, 50 mg, 100 mg, 200 mg, 250 mg, 300 mg, 400 mg, 500 mg, 600 mg, 700 mg or 800 mg.

The compositions of the present invention can comprise combinations of a compound described in this invention and one or more additional therapeutic or prophylactic agents. In this case, the compound can be present in proportions of about 10 to 100% of the dosage normally administered in a monotherapy regimen.

Additional combined therapeutic or prophylactic agents include, but are not limited to, remdesivir, AT-527, interferon, interferon-pegylate, ribavirin, acyclovir, cidofovir, docosanol, famciclovir, foscarnet, fomivirisen, ganciclovir, idoxuridine, penciclovir, trifluridine, valacyclovir, zanamivir, peramivir, imiquimod, lamivudine, zidovudine, didanosine, stavudine, zalcitabine, abacavir, nevirapine, efavirenz, delavirdine, saquinavir, indinavir, ritonavir, nelfinavir, amprenavir, quirky, lprinavir, lopinavir, lopinavir, telaprevir, favipiravir, palivizumab, ombitasvir, pibretasvir, beclabuvir, dasabuvir, daclstasvir, raltegravir, dolutegravir other viral polymerase inhibitors, other RNA-dependent RNA polymerase inhibitors and monoclonal or polyclonal antibodies.

Additional therapeutic agents can be combined with the compounds of this invention to be dispensed in a single dosage form or in a multiple dosage.

In another aspect, the pharmaceutical composition of the present invention further comprises a therapeutically effective amount of one or more immunomodulatory agents as an antiviral agent against coronavirus, in especial SARS-COV-2. Examples of additional immunomodulatory agents include, but are not limited to, alpha, beta, gamma interferons and pegylated form, glucocorticoids, corticoids, dexchlorpheniramine and promethazine.

The pharmaceutical composition of the present invention further comprises a therapeutically effective amount of one or more antibiotics: amikacin, gentamicin, kanamycin, ncomycin, netilmicin, tobramycin, paromomycin, streptomycin, spectinomycin(bs), ansamycins, geldanamycin, herbimycin, rifaximin, carbacephem, loracarbef, carbapenems, ertapenem, doripenem, imipenem/cilastatin, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren,cefoperazone, cefotaxime, cefpodoxime, ceftazidime, ceftibuten, ceftizoxime, moxalactam, ceftriaxone, cefepime, ceftobiprole, teicoplanin, vancomycin, telavancin, dalbavancin, oritavancin, clindamycin, lincomycin, lipopeptide, daptomycin, azithromycin, clarithromycin,crythromycin, roxithromycin, telithromycin, spiramycin, fidaxomicin, monobactams, aztreonam, nitrofurans, furazolidone, nitrofurantoin(bs), oxazolidinones(bs), linezolid, posizolid, radezolid, torezolid, penicillins, amoxicillin, ampicillin, azlocillin, dicloxacillin, flucloxacillin, mezlocillin, methicillin, nafcillin, oxacillin, penicillin g, penicillin v, piperacillin, penicillin g, temocillin, ticarcillin, amoxicillin/clavulanate, ampicillin/sulbactam, piperacillin/tazobactam, ticarcillin/clavulanate, polypeptides, bacitracin, colistin, polymyxin b, quinolones/fluoroquinolones, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, sulfonamides(bs), mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (co-trimoxazole) (tmp-smx), sulfonamidochrysoidine (archaic), tetracyclines(bs), demeclocycline, doxycycline, metacycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol(bs), fosfomycin, fusidic acid, metronidazole, mupirocin, platensimycin, quinupristin/dalfopristin, thiamphenicol, tigecycline(bs), tinidazole, trimethoprim(bs).

The present composition may also contain inactive substances such as dyes, dispersants, sweeteners, emollients, antioxidants, preservatives, pH stabilizers, flavorings, among others, and their mixtures.

In addition, the composition of the present invention may be presented in solid form preferably as a tablet or capsule and in liquid form, preferably as a suspension, solution or syrup, formulated or not with the following components: polyethylenoglicol, Leuprolide acetate and polymer (PLGH (poly (DL-Lactide-coglycolide)), Poly(allylamine hydrochloride), Liposomes, Liposome-proteins SP-B and SP-C and micelles.

The present composition can be administered to children, adults, pregnant women and individuals with mild to severe symptoms of COVID-19, infected with SARS-CoV-2, or other coronavirus potentially exposed or at risk of exposure to SARS-CoV-2, orally or systemically.

The invention further comprises the use of inhibitors of viral RNA synthesis by nitrogenous bases, nucleoside and nucleotide analogs, their derivatives, or salts, solvates, or prodrugs of such compounds, or the compositions of the present invention, for the manufacture of medicine for prophylactic, curative or mitigative treatment for coronavirus, in especial SARS-COV-2 infection, and for the treatment of patients and individuals with, potentially exposed or at risk of COVID-19.

Also disclosed herein is the use of the antiviral compounds and antiviral pharmaceutical compositions, their polymorphs, of the present invention for the manufacture of medicaments to inhibit the action of the coronavirus, in especial SARS-COV-2, replication complex.

Aforementioned medications may additionally comprise one or more antiviral or immunomodulatory compounds for prophylactic, curative or mitigating treatment for coronavirus, in especial SARS-COV-2, infection and for the treatment of individuals potentially exposed to COVID-19. In addition, such medication may comprise from 1 to 3,000 mg of the antiviral compound, preferably from 1 to 500 mg. More preferably 10 mg, 15 mg, 25 mg, 30 mg, 40 mg, 50 mg, 100 mg, 200 mg, 250 mg, 300 mg, 400 mg, 500 mg, 600 mg, 700 mg or 800 mg.

The antiviral compounds of the present invention can be used in the prophylactic, curative or mitigative treatment of individuals infected at the same time by coronavirus, in especial SARS-COV-2, and other viral agents. In addition, such medication may comprise from 1 to 3,000 mg of the antiviral compound, preferably from 1 to 500 mg. More preferably 10 mg, 15 mg, 25 mg, 30 mg, 40 mg, 50 mg, 100 mg, 200 mg, 250 mg, 300 mg, 400 mg, 500 mg, 600 mg, 700 mg or 800 mg.

The antiviral compound of the present invention can be used in the prophylactic, curative or mitigative treatment of individuals infected at the same time by coronavirus, in especial SARS-COV-2, and other viral agents.

In particular, the use of compounds/compositions of the invention for the manufacture of medications to prophylactically, curatively or mitigative the infection associated with coronavirus, in especial SARS-COV-2, and to treat individuals potentially exposed to COVID-19 is directed to pregnant women, elderly and individuals with more aggressive manifestations of infections.

More specifically, the present invention encompasses the use of the cytokinins, such as zeatin, zeatin riboside, kinetin, kinetin riboside, and kinetin riboside monophosphoramidate for the manufacture of pharmaceutical products to prophylactically, curatively or mitigate the infection associated with coronavirus, in especial SARS-CoV-2, of an individual infected with this virus or potentially exposed to it.

Furthermore, the present invention comprises a method of prophylactic, curative (therapeutic) or mitigative treatment of an individual infected with coronavirus, in especial SARS-COV-2, or potentially exposed to this virus, which comprises administering to the individual a combination of the aforementioned compound according to the present invention and one or more antiviral compounds and/or immunomodulators and/or antibiotics.

The treatment methods of the present invention can be administered orally, systemically, intranasally, to individuals infected or preventively potentially exposed to coronavirus, in especial SARS-COV-2.

For oral administration, the composition of the present invention can be formulated in unit dosage forms such as syrup, capsules, tablets or pills, cach containing a predetermined amount of the active ingredient, ranging from about 1 to about 3,000 mg, preferably from 1 to 500 mg, more preferably 10 mg, 15 mg, 25 mg, 30 mg, 40 mg, 50 mg, 100 mg, 200 mg, 250 mg, 300 mg, 400 mg, 500 mg, 600 mg, 700 mg or 800 mg, in pharmaceutically acceptable excipients, including polyethylenoglicol, Leuprolide acetate and polymer (PLGH (poly (DL-Lactide-coglycolide)), Poly(allylamine hydrochloride), Liposomes, Liposome-proteins SP-B and SP-C, micelles.

For parenteral administration, the composition of the present invention can be administered by intravenous, subcutaneous, intranasal or by intramuscular injection. For administration by injection or intranasal, compositions with the compounds in the solution in a sterile aqueous excipient are preferred, which may likewise contain other solutes such as buffers or preservatives, as well as sufficient amounts of pharmaceutically acceptable salts or glucose to prepare the isotonic solution.

Suitable pharmaceutical acceptable vehicles, carriers or excipients that can be used for the aforementioned compositions are described in pharmaceutical texts, for example, in Remington's, The Science and Practice of Pharmacy, 21^{st} edition, 2005 or in Ansel's Pharmaceutical Dosage Forms and Drugs Delivery Systems, 9^{th} edition, 2011.

The dosage of the compound will vary depending on the form of administration and the active ingredient selected. In general, the compound described in this invention is administered in a dose that allows effective antiviral results, however, avoiding any unwanted or harmful side effects.

For oral administration, the compound described in this invention can be administered in the range of about 0.01 to about 3,000 mg per kilogram of body weight per day, preferably from 0.03 to 600 mg, more preferably from 0.05 to 400 mg.

Still as a preferred range can be cited from about 0.05 to about 100 mg per kilogram per day. For systemic administration, the compound described in this invention can be administered in a dosage of about 0.01 to about 100 mg per kilogram of body weight per day, however, attention should be paid to the individual peculiarities of each patient. In a desirable model, the dosage can be in the range of about 0.05 mg to about 50 mg per kilogram of body weight per day, according to the individual peculiarities of cach patient.

The antiviral pharmaceutical composition of the present invention can be used in the therapeutic cure or mitigation of illness in individuals infected at the same time by SARS-COV-2 and other viral agents.

The present invention is described in detail through the examples presented below. It is necessary to emphasize that the invention is not limited to these examples, but also includes variations and modifications within the limits in which it can be developed.

### EXAMPLES

The methods and conditions used in these examples, and the actual compounds prepared in these examples, are not meant to be limiting, but are meant to demonstrate how the compounds can be prepared. Starting materials and reagents used in these examples, when not prepared by a procedure described herein, are generally either commercially available, or are reported in the chemical literature, or may be prepared by using procedures described in the chemical literature.

### Example 1

### Preparation of High-Purity Grade of MB-905 (Kinetin)

A 20 L reactor was charged with 7.5 L of ethanol and 1.5 Kg of 6-chloropurine (9.7 mol, 1.0 equiv.). A solution of furfurylamine (1.8 L, 20.4 mol, 2.1 equiv.) and 4-dimethylaminopyridine (11.9 g, 0.097 mol, 0.01 equiv.) in 250 mL of ethanol was added dropwise to the solution of 6-chloropurine. The mixture was heated to 85° C. and stirred for 4 h. After reaction completion, the mixture was cooled to 5° C. and stirred for 40 min. The crystals were filtered and washed with 3.0 L of ethanol. Next, the crystals were transferred to 20 L reactor with 4.5 L of a mixture of ethanol and water 1:1. The suspension was stirred for 30 min. The crystals were filtered, washed with 1.5 L of ethanol and dried at 65° C. for 12 h under vacuum to give crude Kinetin (1.92 kg).

For high-purity grade of kinetin, the crude kinetin was purified by following steps described below:

A 20 L reactor was charged with 9.35 L of ethanol and 1.87 Kg of crude Kinetin (8.69 mol, 1.0 equiv.). A solution of HCl (750 mL, 9.08 mol, 1.04 equiv.) in 1.87 L of distilled water was added dropwise to the solution of Kinetin. The mixture was heated to 75° C.and stirred until complete dissolution of the solid. Then, 19 g of activated charcoal was added to the solution, and the mixture was stirred for 15 min at 75° C. The mixture was filtered over celite and washed with 1.87 L of hot ethanol. Then, the resulting filtrate solution was cooled to 10° C. and stirred for 1 h. The crystals were filtered and washed with 940 mL of cooled ethanol. After this step, the crystals were suspended in 1.87 L of cooled ethanol and stirred for 15 min. The crystals were again filtered and washed with 940 mL of cooled ethanol. The crystals were suspended in 4.67 L of solution ethanol:water 1:1, stirred and cooled to 10° C. Then, 1.18 L of tricthylamine was added dropwise to the mixture and stirred for 30 min at 10° C. The solid was filtered and washed with 1.87 L of the mixture of ethanol and water 1:1. After this step, the solid was suspended in 4.68 L of distilled water and stirred for 30 min. Next, the solid was filtered and washed successively with, 1.87 L of distilled water, 1.87 L of ethanol: water 1:1 and 1.87 L of ethanol.

The solid was dried to constant weight under vacuum at 65° C., to give high-purity grade of Kinetin as white solid (1.48 kg, 6.88 mol, 71% yield, purity: 99.9 area % HPLC). ¹H NMR (500 MHZ, DMSO-d6) δ 12.99 (s, 1H), 8.20 (s, 1H), 8.11 (s, 2H), 7.54 (s, 1H), 6.35 (s, 1H), 6.22 (s, 1H), 4.69 (s, 2H) ppm; ¹³C NMR (126 MHZ, DMSO-d6) δ 154.0, 153.2, 152.3, 149.7, 141.8, 139.1, 119.0, 110.5, 106.6, 36.5 ppm. IV (λ0 max): 3251, 3203, 3104, 3054, 2982, 2950, 2911, 2825, 2720, 2681, 2565, 1895, 1696, 1625, 1590, 1538, 1503, 1482, 1459, 1439, 1404, 1367,1337, 1327, 1308, 1280, 1253, 1219, 1199, 1157, 1149, 1129, 1069, 1016, 1008, 935, 917, 892, 861, 845, 814,796, 751, 726, 702, 678, 660, 639, 602; m.p. 265-272° C.dec; HPLC (X Terra RP18, 250×4.6×5 mm, isocratic 10% fase B [MeOH:ACN (1:1)]: 90% fase A [3,4 g/L KH₂PO₄ pH 2,54 (H₃PO₄)], 1.0 mL/min, 210 nm, 30° C) r.t=8.768 min, λₘₐₓ 272 nm; HRMS-MALD-TOF/TOF (m/z): Calc. for C₁₀H₁₀N₅O⁺[M+H]⁺216.08799, found 216.08813.

### Example 2

Compound MB-907 can be prepared, for example, according to the procedure illustrated in Scheme 2.

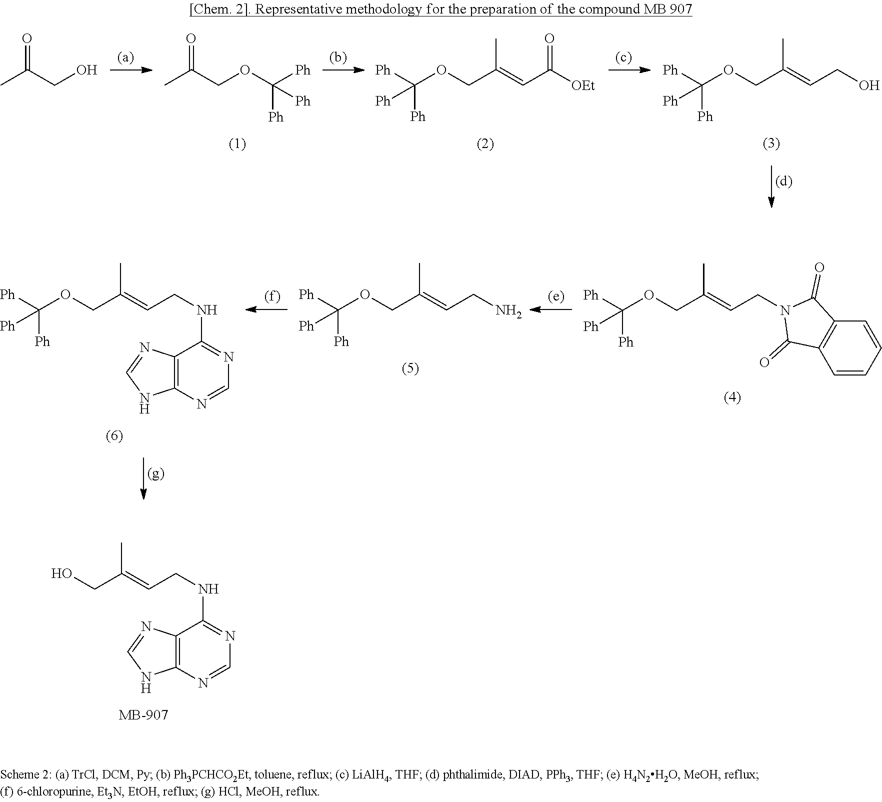

### Example 3

Compound MB-711 can be prepared, for example, according to the procedure illustrated in Scheme 3.

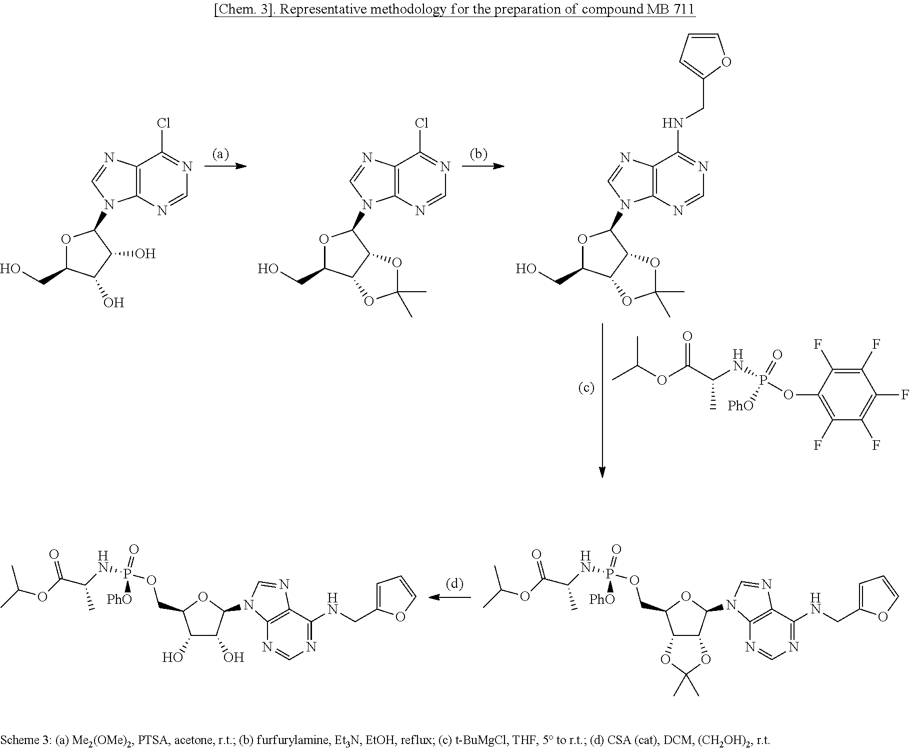

### Example 4

African green monkey kidney cells (Vero), human hepatoma (HuH-7) and Calu-3 cells are permissive to SARS-COV-2 and they grow at high quantitates in the laboratory. Cells were cultured in high glucose DMEM complemented with 10% fetal bovine serum (FBS; HyClone, Logan, Utah), 100 U/mL penicillin and 100 μg/mL streptomycin (Pen/Strep; ThermoFisher) at 37° C. in a humidified atmosphere with 5% CO2. Thus, they represent suitable models for screening of compounds with biological activity. Cells were infected at multiplicities of infection (MOI) of 0.01 to 0.5. Cultures were treated after 1 h of infection. At 24 h (Vero) and 48-72 h (Huh-7 and Calu-3) cells were lysed, and cell-associated viral RNA quantified. The total viral RNA from culture supernatants was extracted. Quantitative RT-PCR was performed using one-step Real-Time PCR System reaction with primers, probes, and cycling conditions recommended by the Centers for Disease Control and Prevention (CDC) protocol were used to detect the SARS-COV-2.

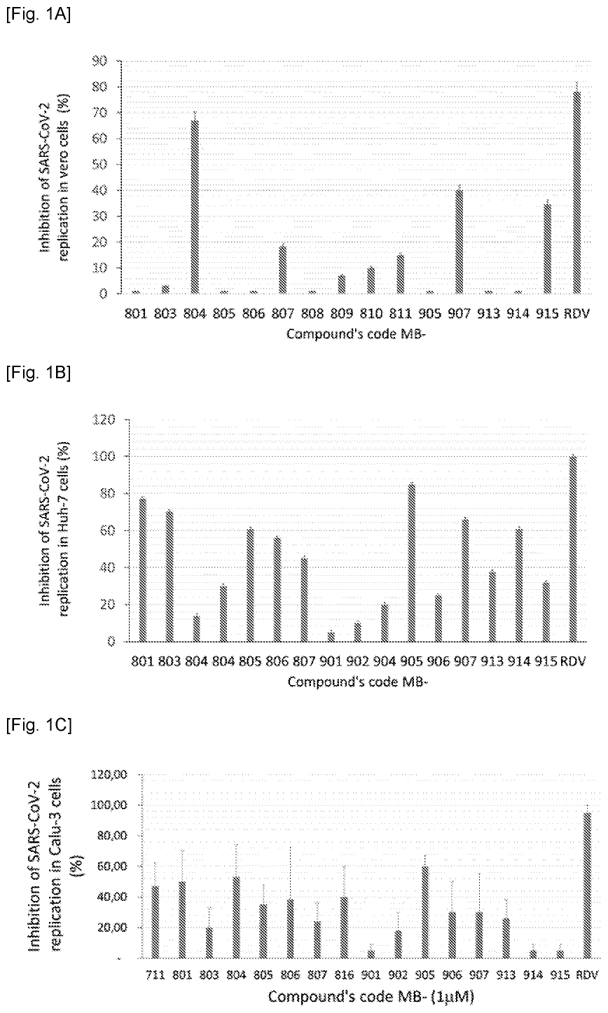

We found that among the tested compounds to inhibit SARS-COV-2 replication in Vero cells, the compounds MB-804 and MB-907 produced the best inhibitory profiles, showing the ranging from 40 to 70% inhibition of viral RNA synthesis at 1.0 μM ([FIG. 1A]). In Huh-7 cells, a more versatile system to allow entry of nitrogenous bases, nucleoside and nucleotides into biochemical pathways, more substances displayed good profile to affect the viral RNA synthesis, inhibitory activity ≥50% at 1.0 μM ([FIG. 1B]), such as compounds MB-801, MB-803, MB-805, MB-806, MB-807, MB-905, MB-907 and MB-914. To inhibit SARS-COV-2 RNA synthesis in calu-3 cells, MB-711, MB-801, MB-803, MB-805 and MB-905 showed the best profiles at 1.0 μM ([FIG. 1C]). These data demonstrate that SARS-COV-2 RNA synthesis is inhibited by nitrogenous bases, nucleoside and nucleotides analogs described here.

### Example 5

The pharmacological parameters to inhibit SARS-COV-2 RNA synthesis and productive replication were characterized for the most active compounds that emerged from the initial screening. Cell-associated Viral RNA synthesis was characterized, as described in the example 1, in Vero and Huh-7 cells infected at MOIs of 0.01 and 0.1, respectively. Treatments were performed in a single moment, after 1 h of inoculation. Remdesivir (RDV) and MK-4482 were used as positive controls.

To confirm that the inhibitory activity at the level of SARS-COV-2 RNA synthesis represented a real ability to suppress viral replication in cellular systems relevant to the physiopathology of COVID-19, we challenged calu-3 type II human pneumocytes with SARS-COV-2 at MOI of 0.5. Treatments were performed in a single moment, after 1 h of inoculation, or daily. After 48-72 h, culture supernatant was harvested and the infectious virus titers determined by titration in Vero cells. After infection with supernatant from Calu-3 cells, Vero cells were overlayed with fresh semi-solid medium containing 2.4% of carboxymethylcellulose (CMC) was added and culture was maintained for 72 h at 37° C. Cells were fixed with 10% Formalin for 2 h at room temperature and then, stained with crystal violet (0.4%). Therefore, we measured if the virus progeny grown in the presence of the compounds would have a limited ability to perform a subsequent round of infection.

In parallel, cytotoxicity assays were performed. Monolayers of 1.5×10⁴ cells in 96-well plates were treated for 3 days with various concentrations (semi-log dilutions from 1,000 to 10 μM) of the antiviral drugs. Then, 5 mg/mL 2,3-bis-(2-methoxy-4-nitro-5-sulfophenyl)-2H-tetrazolium-5-carboxanilide (XTT) in DMEM was added to the cells in the presence of 0.01% of N-methyl dibenzopyrazine methyl sulfate (PMS). After incubating for 4 h at 37° C., the plates were measured in a spectrophotometer at 492 nm and 620 nm.

Cytokinins (MB-905) and MB-907, and nucleoside MB-801, showed 10 to 80-times higher potencies to inhibit SARS-COV-2 RNA synthesis in huh-7 than Vero Cells—meaning that human cells are more prompt to active these compounds (Table 2). Similarly, MK-4482 also gained potency to inhibit viral replication in huh-7 hepatoma cells. One exception is the nucleoside MB-804, which displayed better in vitro potency to inhibit virus RNA synthesis in Vero cells (Table 2). There was at least a 10-fold difference in favor of RDV to inhibit viral RNA synthesis compared to our compounds (Table 2). On the other hand, the MB potencies in Vero and huh-7 cells were comparable to MK-4482 (Table 2). Moreover, because the MBs are less cytotoxic than MK-4482. our best compounds displayed selectivity indexes (SI) orders of magnitude superior to MK-4482 (Table 2), meaning that they have safer margins for in vitro use than this reference compound. Our compounds in general presented half of the RDV's cytotoxicity as shown in Table 2.

TABLE 2
Summary of the in vitro parameters of selected compounds

| | | | | | | | Calu-3 | | | | Calu-3 | | | |
|---|---|---|---|---|---|---|---|---|---|---|---|---|---|---|
| Cells and | Vero | | | Huh-7 | | | (MOI 0.5 72 h) | | | | (MOI 0.5 72 h) | | | |
| experimental | (MOI 0.01 24 h) | | | (MOI 0.1 48 h) | | | Single moment treatment | | | | Daily treatment | | | |
| conditions | EC₅₀ | CC₅₀ | | EC₅₀ | CC₅₀ | | EC₅₀ | EC₉₀ | CC₅₀ | | EC₅₀ | EC₉₀ | CC₅₀ | SI |
| Compounds | [μM] | [μM] | SI | [μM] | [μM] | SI | [μM] | [μM] | [μM] | SI | [μM] | [μM] | [μM] | [μM] |
| Remdesivir | 0.7 ± | 530 ± | 757 | 0.006 ± | 280 ± | 46666 | 0.15 ± | 0.4 ± | 350 ± | 2300 | 0.01 ± | 0.2 ± | 330 ± | 33000 |
| | 0.1 | 80 | | 0.002 | 50 | | 0.03 | 0.1 | 50 | | 0.003 | 0.01 | 40 | |
| EIDD-2801 | NT | NT | ND | INT | NT | ND | | | | | 0.02 ± | 8.6 ± | 58 ± | 2900 |
| | | | | | | | | | | | 0.008 | 0.2 | 18 | |
| MB 711 | NT | NT | ND | NT | NT | ND | 1.1 ± | 9.2 ± | 562 ± | 510 | 0.01 ± | 3.8 ± | 580 ± | 58000 |
| | | | | | | | 0.03 | 0.1 | 46 | | 0.008 | 0.3 | 72 | |
| MB 801 | 8 ± | NT | ND | 0.1 ± | 229 ± | 2290 | 0.18 ± | 7.2 ± | 550 ± | 3055 | 0.02 ± | 3.7 ± | 530 ± | 26500 |
| | 2 | | | 0.02 | 28 | | 0.02 | 0.5 | 32 | | 0.003 | 0.2 | 65 | |
| MB-905 | 5 ± | NT | ND | 0.1 ± | 560 ± | 5600 | 0.31 ± | 2.8 ± | 620 ± | 2000 | 0.03 ± | 2.1 ± | 538 ± | 17933 |
| | 1.5 | | | 0.02 | 40 | | 0.05 | 0.3 | 80 | | 0.004 | 0.3 | 64 | |
| MB 907 | 6 ± | NT | ND | 0.05 ± | 600 ± | 12000 | NT | NT | NT | ND | NT | NT | NT | ND |
| | 1.8 | | | 0.03 | 180 | | | | | | | | | |
| MB 804 | 1.4 ± | 636 ± | 454 | 7.2 ± | 550 ± | 78 | 1.2 ± | 9.1 ± | 580 ± | 500 | NT | NT | NT | ND |
| | 0.15 | 50 | | 1.2 | 18 | | 0.1 | 0.9 | 20 | | | | | |

NT-not tested, ND-not determined, EC₅₀-(i.e. the concentration of tested compound necessary to reduce by 50% the number of viral plaque formed in a monolayer of cells in a fixed period of time incubation relative to virus grown in the absence of test compound), EC₉₀-inhibitory activity by 90%, CC₅₀-cytotoxic concentration by 50%, SI-selectivity index (calculated by the ratio of CC₅₀/EC₅₀).

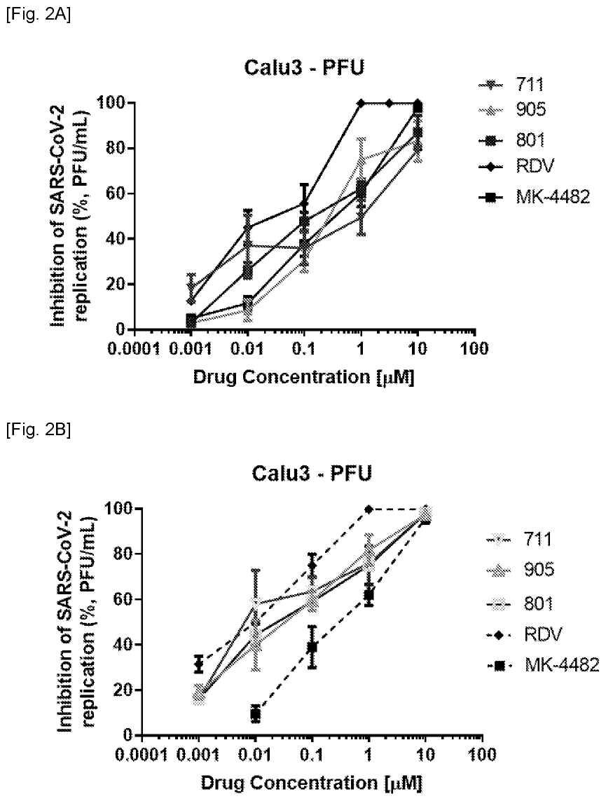

To inhibit productive replication if the SARS-COV-2 in Calu-3 type II pneumocytes, RDV displayed a decreased potency compared to its antiviral activity in Huh-7 hepatoma cells in a single moment treatment scheme (Table 2). It is inventive that differently than RDV, the MBs potency did not change substantially to inhibit virus replication in these cells (Table 2). MBs' low cytotoxicity and potency at the submicromolar range, in a single moment treatment scheme, rendered to these investigated compounds SI values comparable or superior to the reference compounds—RDV and MK-4482, respectively (Table 2). Of note, MBs displayed efficiency bellow 10 μM to inhibit SARS-COV-2 replication in Calu-3 cells, when compared to MK-4482 (Table 2). MB-905 was the most potent among the candidates, with EC₉₀ equals to 2.8 μM (Table 2). Inhibitory concentration response curves highlight the antiviral performance of the nitrogenous base MB-905, the nucleoside MB-801 and the nucleotide (monophosphoramidate) MB-711 in comparison to the reference compounds RDV and MK-4481 ([FIG. 2A]).

Next, SARS-COV-2 susceptibility to MBs in Calu-3 cells was tested in a daily treatment scheme. That is, besides the treatment just after inoculation, treatments were repeated in the following days, meaning that cells were treated additionally one or two times. This in vitro treatment scheme could be considered consistent with daily-dose therapy regimen routinely used in patients exposed to infectious diseases. Under these experimental conditions, all the molecules, either MBs or RDV, displayed potency and efficiency, respectively of 10- and 2-times higher than the single moment treatment (Table 2). This approach reduced the differences in the concentration-dependent inhibition curve compared to RDV ([FIG. 2B] and Table 2). Neither RDV nor the MBs displayed higher cytotoxicity upon multi-time treatments (Table 2). Conversely, MK-4482 cytotoxicity became higher due to daily treatment, with more cell death MK-4482 efficiency was impaired 2-times (Table 2).

Altogether, our data revel that nitrogenous base MB-905, nucleoside MB-801 and nucleotide MB-711 are endowed with anti-coronavirus activity, as reveled by the prototypic SARS-COV-2 strain, and their in vitro pharmacological parameters became more favorable in representative cells of the respiratory tract.

### Example 6

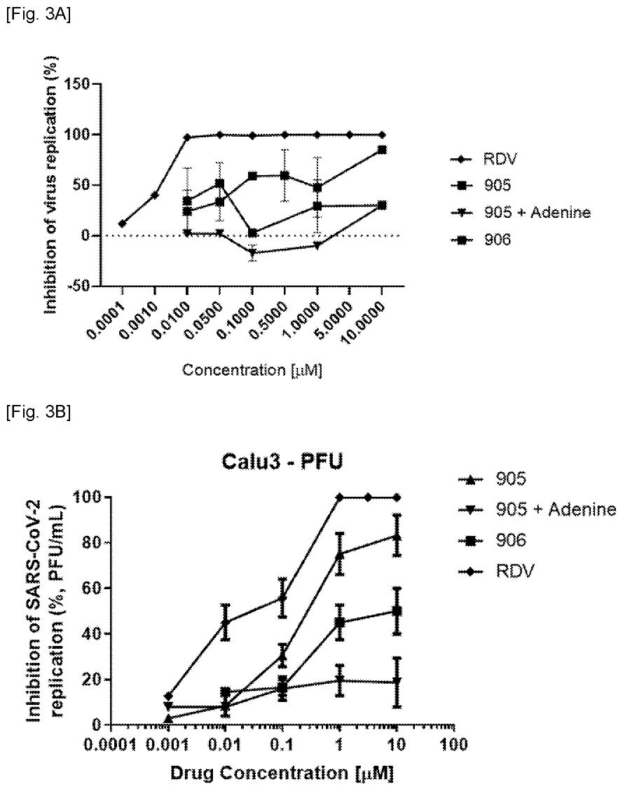

Nucleoside and monophosphate nucleotide analogs endowed with antiviral activity need to be converted to their triphosphate metabolite to become active. It is less frequent the use of nitrogenous bases as antiviral pro-drugs. To confirm that, by structural analogy with adenine, the MB-905 would enter into the cellular metabolism through the adenine phosphoribosyl transferase (APRT) experiments described in example 2 were performed in the presence of adenine (as competitor base) or with an analog of MB-905 blocked in the position 9 (MB-906). Both in huh-7 and in calu-3 cells, MB-905 treatment just after inoculation produces a concentration dependent inhibition of SARS-COV-2 replication ([FIG. 3A] and B). Simultaneous treatment with adenine at 10 μM prevented MB-905′s anti-coronavirus activity ([FIG. 3A] and B). Moreover, MB-906, which is unable to receive a ribose 5′ phosphate radical at position 9, was not endowed with anti-coronavirus activity ([FIG. 3A] and B).

### Exemple 7

To confirm the rational that the drugs inhibit viral RNA synthesis in physiologically relevant cells, intracellular levels of SARS-COV-2 genomic and subgenomic RNA were measured in type II pneumocytes, Calu-3 cells. Calu-3 cells were infected at MOI of 0.5. After 1 h inoculation period, cells were washed with phosphate saline buffer (PBS) to remove unbounded viruses and treated with 1 μM of the indicated compounds. After 48-72 h after infection, cell monolayers were lysed total viral RNA was extracted, quantitative RT-PCR was performed, to detect genomic (ORF1) and subgenomic (ORFE) were detected, as described elsewhere.

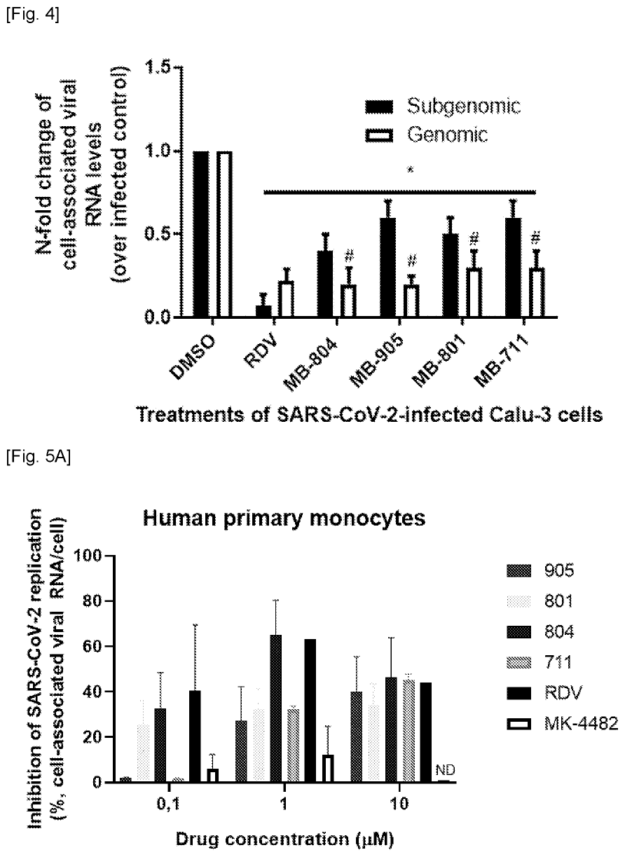

The most active cytokinin and their derivatives inhibited viral RNA synthesis, being more effective to reduce genomic replication than sub-genomic RNA synthesis from 50 to 70%, respectively ([FIG. 4]). For comparison, remdesivir (RDV) was used as positive control.

### Example 8

Human primary monocytes were obtained after 3 h of plastic adherence of peripheral blood mononuclear cells (PBMCs). PBMCs were isolated from healthy donors by density gradient centrifugation (Ficoll-Paque, GE Healthcare). PBMCs (2.0×10⁶ cells) were plated onto 48-well plates (NalgeNunc) in RPMI-1640 without serum for 2 to 4 h. Non-adherent cells were removed, and the remaining monocytes were maintained in DMEM with 5% human serum (HS; Millipore) and penicillin/streptomycin. Monocytes were infected at MOI of 0.1. After 1 h inoculation period, cells were washed with phosphate saline buffer (PBS) to remove unbounded viruses and treated with 1 μM of the indicated compounds. After 24 h after infection, cell monolayers werc lysed total viral RNA was extracted, quantitative RT-PCR was performed, to detect the SARS-COV-2 and the housekeeping gene RNAse P.

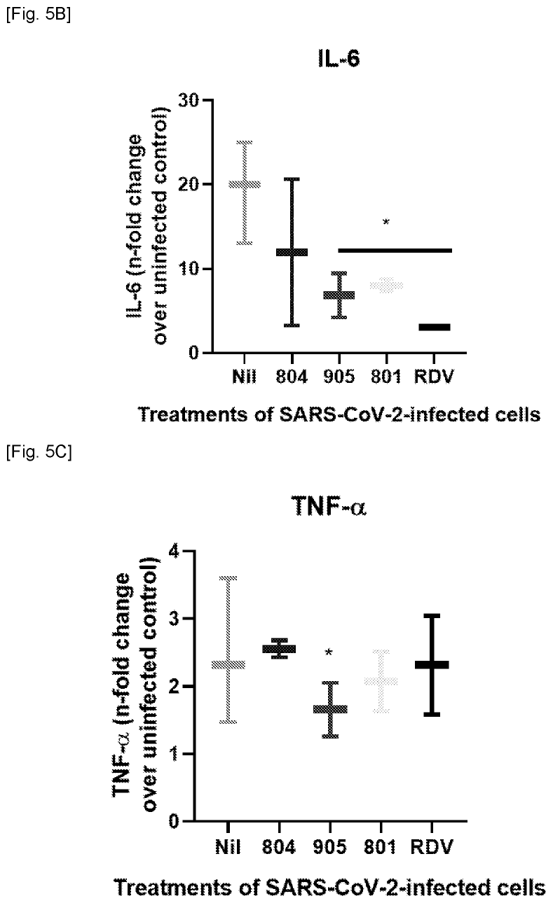

In SARS-COV-2-infected human primary monocytes, compounds MB-905, MB-801, and MB-804 were able to reduced viral RNA levels/cell ([FIG. 5A]). The pair of nitrogenous bases (MB-905) and nucleoside (MB-801) also reduced the SARS-CoV-2-induced enhancement of TNF-α and IL-6 levels in the culture supernatant ( [FIG. 5B] and C). These data provide further evidence for a putative benefit in COVID-19 with the investigated drugs, if target concentrations can be achieved in patients.

### Example 9

### the Antiviral Activity of the Compounds Against SARS-COV-2 Production of Infectious Virus Particles is Enhanced by Co-Inhibition of Exonuclease

It has been demonstrated that SARS-COV-2 exonuclease activity, catalyzed by its dimer nsp14/10, is inhibited by several classes of compounds. In particular, the HIV integrase inhibitor rategravir may impair nsp14 activity. We obtained synergistic results between MB-905, MB 801, MB 711, and MB-804 with either raltegravir or dolutegravir (as inhibitors of exonuclease; iEXO). The one-log (90%) inhibition of viral replication, obtained with the MBs alone, was enhanced to an additional log (FIG. 6), either by raltegravir ([FIG. 6A]) or by dolutegravir ([FIG. 6B]). As a control, sofosbuvir and tenofovir also display enhanced efficiency to inhibit SARS-COV-2 in calu-3 cells in the presence of raltegravir or dolutegravir (FIG. 6). On the other hand, RDV, a delayed-chain terminator, was not affect by iEXO (FIG. 6). Altogether, the results from MBs, sofosbuvir and tenofovir are consistent with the notion that nsp12, the RNA-dependent RNA polymerase, may incorporate them into virus RNA, but nsp14 could remove the modified nucleotides. The exception is RDV, because of nsp12 has a higher affinity for this drug over ATP and its delayed termination, this compound could be more resistant to nsp14 excision.

### Example 10

### MB-905 Affects SARS-COV-2 Codon Usage

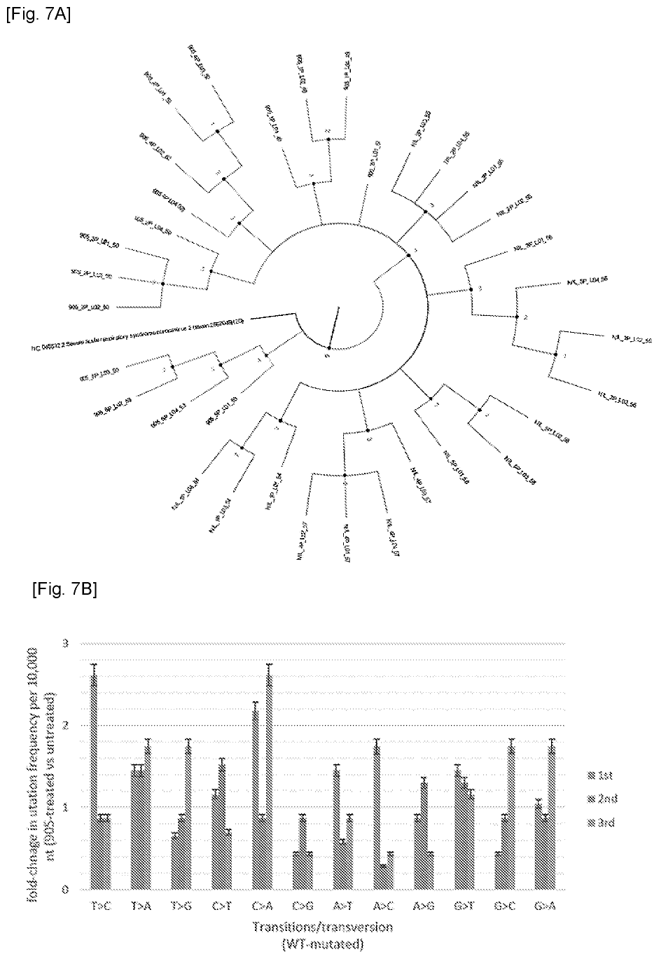

Since we described that MBs can inhibit SARS-COV-2 RNA synthesis and could be removed by exonuclease activity, growth of the virus in the presence of MB-905 could indicate its mechanism of action upon incorporation in the viral RNA. SARS-COV-2 was propagated in the presence and absence of MB-905. After each passage, a new round of propagation was carried out under a higher concentration. Virus RNA in the supernatant was sequenced in a depth consistent to monitor viral sub-population and at error rate below 0.001%. When pondering nucleotide transitions and transversions, SARS-COV-2 sequences generated in the presence of MB-905 segregated from those that grew in the absence of this pressure ([Fig.7A]). There were numerous non-synonymous mutations induced by MB-905, in especial changes T (U)=C and C=A suggest codon biased ([FIG. 7B]). These results are in line with example 6, because MB-905 codon bias could be corrected by exonuclease; thus, inhibition of proofreading activity synergizes with our compound. Moreover, examples 7, 6 and 4, crosstalk towards consistency, because it is more likely that codon bias will scape proofreading activity in larger genetic sequences than in small fragments. Indeed, a more pronounced inhibition of genomic, than sub genomic, RNA synthesis ([FIG. 4]) was observed.

### Example 11

### Pharmacokinetics of MB-905MB-905 in Rodent Plasma

The pharmacokinetic assay was performed by using mice of the CDI strain (20-30 g) or rat Sprague Dawley (250-300 g) of both sexes from the Center of Innovation and Preclinical Studies (CIEnP) vivarium. All animals were maintained under SPF (Specific Pathogen Frec) animal conditions and were obtained from CIEnP facility, whose breeding colonies were purchased from Charles River Laboratories (USA). The pre-formulations used to dissolve MB-905 are as follow: dose of 3 mg/kg (i.v.): 1% DMSO+4% PEG400+0.5% Tween80 e 94.5% Saline, dose of 30 mg/kg (p.o.): 10% DMSO+40% PEG400+5% Tween80 and 45% saline, dose of 550 mg/kg (p.o.): 5% Tween 80+95% PEG400. The trial consisted of administering MB-905 at doses of 10, 30 or 550 mg/kg, orally or with a dose of 3 mg/kg intravenously. After oral or intravenous administration, blood was collected at times of 0.25, 0.5, 1, 2, 4, 8 and 24, while after intravenous administration the collection times were 0.083, 0.25, 0.5, 1, 2 and 4 hours. The samples collected from each animal and at each collection time were processed and analyzed individually. For the analysis of plasma and lung, UPLC-MS/MS equipment was used, whose system consists of a Xevo TQS mass spectrometer with a triple-quadrupole mass analyzer, from Waters. The mass spectrometer is coupled to a high-performance liquid chromatograph (Acquity H-Class). The acquisition and treatment of the data were performed with the MassLynx software. The pharmacokinetic parameters evaluated were: AUC (AUC 0-T or, AUC 0-∞), Cₘₐₓ, Tₘₐₓ, T_{1/2}, volume of distribution, clearance, elimination constant and bioavailability. The calculation of bioavailability was performed using the following equation: F (%)=[(Intravenous dose×oral AUC)/(Oral dose×Intravenous AUC)]×100.

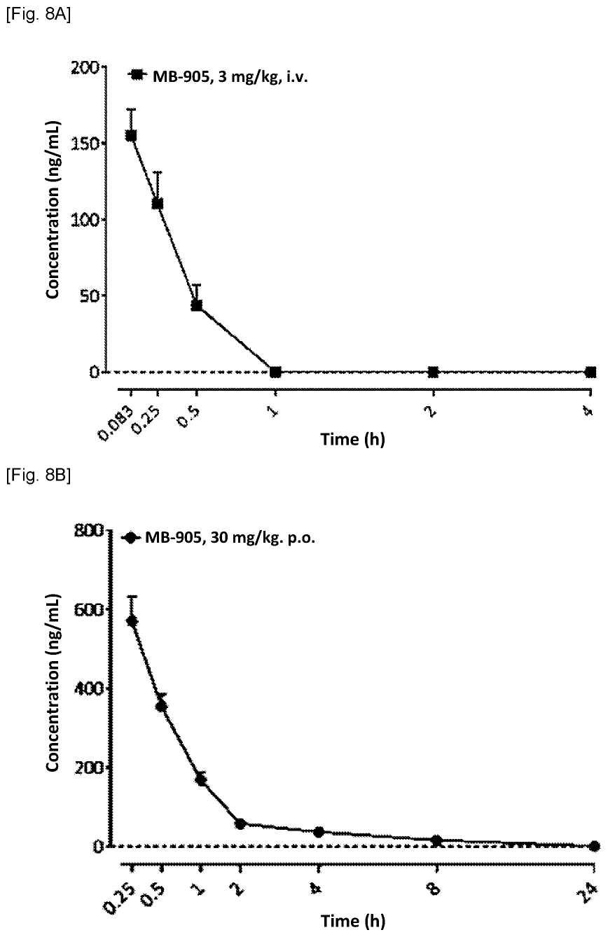

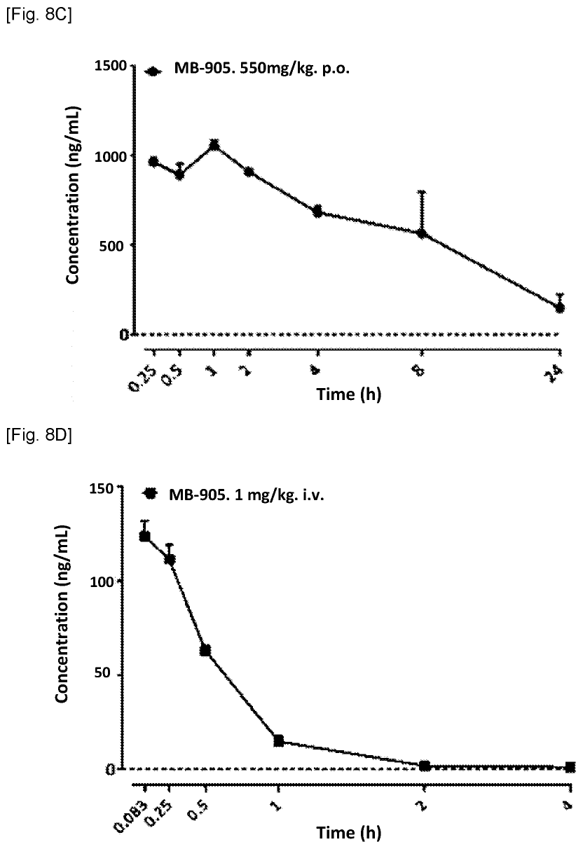

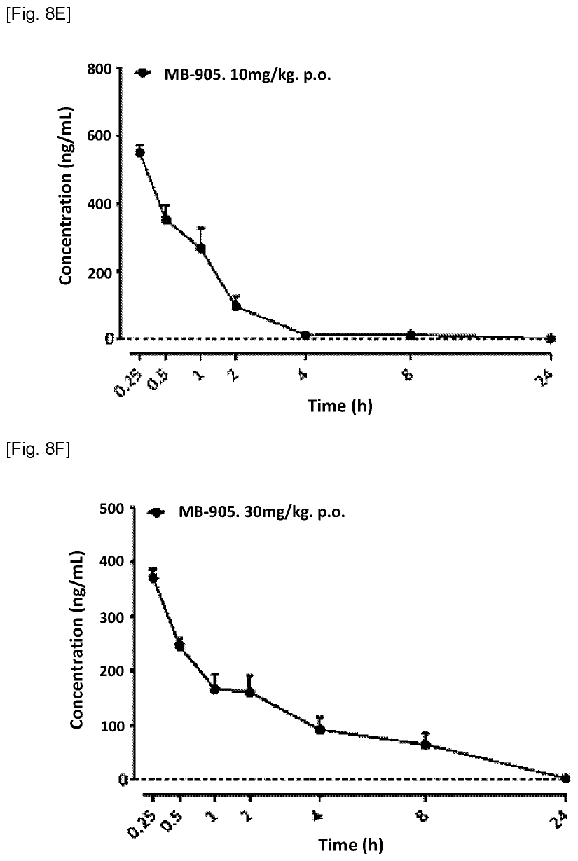

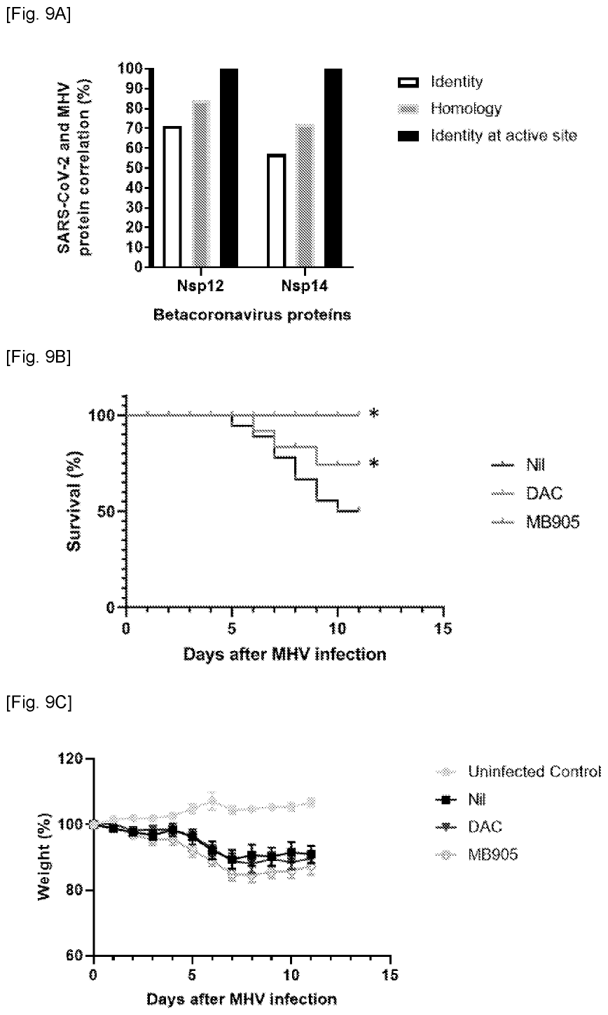

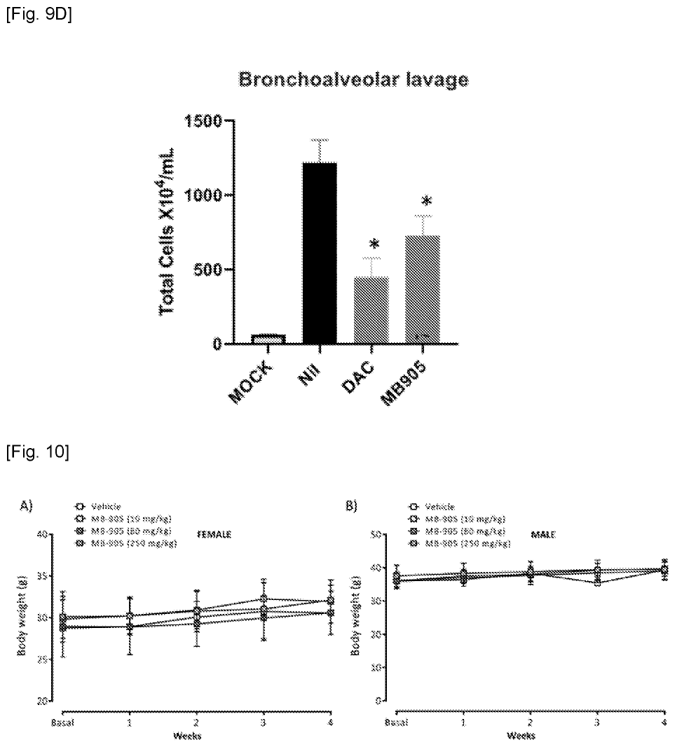

After systemic administration of compound MB-905 given intravenously to mice (3 mg/kg) or orally (30 mg/kg) the peak plasma concentrations were 135.16 and 569.97 ng/mL, time reach maximal concentrations of 0.083 and 0.083 hour, time of half-life of 0.22 and 1.1 hour, Volume of distribution of 19.40 and 102.21 L/kg, clearance of 918.38 and 1060.15 mL/min/kg, area under de curve (last) of 55.58 and 365.63 h ng/mL, area under de curve (all) of 66.41 and 355.63 h ng/mL, elimination rate constant of 3.09 and 0.62 1/h, respectively. The bioavailability of MB-905 in mice was estimated as being 53,5% (FIGS. 8A-B and Table 3). When MB-905 was given orally to mice in very high dose (550 mg/kg,) the NOAEL (Non Observable Adverse Event Level) the pharmacokinetic parameters were: the peak plasma concentration of 1,053.37 ng/mL, time to reach maximal concentration of 0.5 hour, clearance 1,843.18 mL/min/kg, time of half-life of 2.72 hours, volume of distribution of 1,843.18 L/kg, area under de curve (last) of 4,392.27 h ng/mL, area under de curve (all) of 4,392.27 h ng/mL, elimination rate constant of 0.25 1/h, respectively. The bioavailability of MB-905 was 36.1% ([FIG. 8C] and Table 3). Importantly, the in vitro pharmacological parameters for MB-905 in human cells ranged from 0.1 to 2.8 μM (Table 2), which are respectively equivalent to 21.5 to 602 ng/ml (molecular weight of 215 g/mol). In light of the pharmacokinetics and the NOAEL, plasma exposure is consistent with doses required to achieve anti-coronavirus activity.

TABLE 3
Pharmacokinetic parameters of MB-905 in mice

| | Pharmacokinetic parameters in plasma of mice | | | | | | | | | |
|---|---|---|---|---|---|---|---|---|---|---|
| | Dose | | | | CL | | AUCₗₐₛₜ | AUCₐₗₗ | | |
| | and | Cₘₐₓ | Tₘₐₓ | T_{1/2} | (mL/ | Vz | (h*ng/ | (h*ng/ | Ke | F |
| Compound | Route | (ng/mL) | (h) | (h) | min/kg) | (L/kg) | mL) | mL) | (1/h) | (%) |
| MB-905 | 3 mg/kg | 155.16 | 0.083 | 0.22 | 918.38 | 19.04 | 55.48 | 66.41 | 3.09 | 100% |
| | (i.v.) | | | | | | | | | |
| MB-905 | 30 mg/kg | 569.97 | 0.083 | 1.11 | 1060.15 | 102.21 | 355.63 | 355.63 | 0.62 | 53.5% |
| | (v.o.) | | | | | | | | | |
| MB-905 | 550 mg/kg | 1053.37 | 0.5 | 2.72 | 1843.18 | 434.20 | 4392.27 | 4392.27 | 0.25 | 36.1% |
| | (v.o.) | | | | | | | | | |

Cₘₐₓ: Peak concentration; Tₘₐₓ: Time to reach Cₘₐₓ; T_{1/2}: half-life; CL: Clearance; Vz: Volume of distribution; AUCₗₐₛₜ: Area under de curve (last); AUCₐₗₗ area under de curve (all); Ke: elimination rate constant; F: bioavailability;

When given to rats by intravenously route MB-905 (3 mg/kg) the pharmacokinetic obtained parameters were: peak plasma concentration of 99.37 ng/mL, time to reach maximal concentration of 0.25 hour, time of half-life of 0.11 hour, volume of distribution of 13.43 L/kg, clearance of 1,336.77 mL/min/kg, area under de curve (last) of 34.72 h ng/ml, area under de curve (all) of 35.20 h ng/mL, elimination rate constant of 0.25 1/h, respectively ([FIG. 8D] and Table 4).

TABLE 4
Pharmacokinetic parameters of MB-905 in rats

| | Pharmacokinetic parameters in plasma of the rat | | | | | | | | | |
|---|---|---|---|---|---|---|---|---|---|---|
| | Dose | | | | CL | | AUCₗₐₛₜ | AUCₐₗₗ | | |
| | and | Cₘₐₓ | Tₘₐₓ | T_{1/2} | (mL/ | Vz | (h*ng/ | (h*ng/ | Ke | F |
| Compound | Route | (ng/mL) | (h) | (h) | min/kg) | (L/kg) | mL) | mL) | (1/h) | (%) |
| MB-905 | 3 mg/kg | 123.61 | 0.083 | 0.56 | 213.63 | 10.40 | 77.12 | 77.12 | 1.23 | 100% |
| | (i.v.) | | | | | | | | | |
| MB-905 | 10 mg/kg | 549.96 | 0.25 | 1.45 | 241.09 | 30.37 | 666.14 | 761.91 | 0.47 | 98.8% |
| | (v.o.) | | | | | | | | | |
| MB-905 | 30 mg/kg | 370.47 | \|0.25 | 3.81 | 330.53 | 109.18 | 1498.08 | 1498.08 | 0.18 | 64.7% |
| | (v.o.) | | | | | | | | | |

Cₘₐₓ: Peak concentration; Tₘₐₓ: Time to reach Cₘₐₓ; T_{1/2}: half-life; CL: Clearance; Vz: Volume of distribution; AUCₗₐₛₜ: Area under de curve (last); AUCₐₗₗ area under de curve (all); Ke: elimination rate constant; F: bioavailability;

When given orally to rats (10 and 30 mg/kg) MB-905 was well absorbed with the following pharmacokinetic parameters: peak plasma concentrations of 544.96 and 370.47 ng/mL, time to reach maximal plasma concentrations of 0.25 and 0.25 hour, time of half-life of 1.46 and 3.81 hours, volume of distributions of 30.37 and 109.18 L/kg, clearance of 241.09 and 330.57 mL/min/kg, area under de curves (last) of 666.14 and 1,498.09 h ng/mL, area under de curve (all) of 761.91 and 1,498.09 h ng/mL, elimination rate constant of 0.47 and 0.18 1/h, respectively. The bioavailability of MB-905 in rats was estimated as being 98.8 and 64.7% for the doses of 10 and 30 mg/kg, respectively (FIGS. 8E and 8F and Table 4).

### Example 12

### Pharmacokinetics of MB-905 in Three Different Formulations

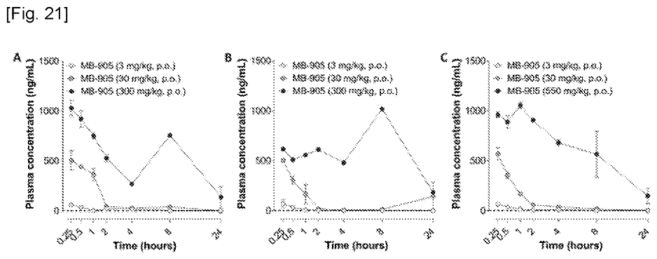

To evaluate the pharmacokinetic profile with different pre formulations, mice were orally treated with MB-905 (3, 30 and 300 mg/kg) with compound pre formulated with 5% tween80+95% PEG400 (FIG. 21A and table 5) or 5% carboxymethylcellulose (FIG. 21B and table 5) and MB-905 (3, 30 and 550 mg/kg) in 5% ethanol, 30% Propylene Glycol, 45% polyethylene glycol 400 (PEG 400) and 20% water (FIG. 21C and table 5). All preformulation showed similar results with Cmax, but, the half-life (T1/2) was better with dose of 30 mg/kg of MB-905 when diluted in 5% ethanol, 30% propylene glycol, 45% polyethylene glycol 400 (PEG 400) and 20% water), than compared with tween80+95% PEG400 or 5% carboxymethylcellulose. Thus, the pre-formulation was used for most subsequent in vivo studies.

TABLE 5
Pharmacokinetic parameters of MB-905 in mice

| | | | | | | | 30% Propylene MB-905 | | |
|---|---|---|---|---|---|---|---|---|---|
| | | | | | | | (5% ethanol, Glycol, | | |
| | | | | | | | 45% polyethylene glycol | | |
| | MB-905 | | | MB-905 | | | 400 (PEG 400) | | |
| | (5% Tween80 + 95% PEG400) | | | (5% Carboxymethylcellulose) | | | and 20% water) | | |
| Dose and | 3 mg/kg | 30 mg/kg | 300 mg/kg | 3 mg/kg | 30 mg/kg | 300 mg/kg | 3 mg/kg | 30 mg/kg | 550 mg/kg |
| Route | (p.o.) | (p.o.) | (p.o.) | (v.o.) | (p.o.) | (p.o.) | (v.o.) | (p.o.) | (p.o.) |
| Cₘₐₓ ng/mL) | 60.84 | 506.92 | 1030.34 | 70.18 | 503.62 | 1018.72 | 63.95 | 569.97 | 1053.37 |
| Tₘₐₓ (h) | 0.25 | 0.25 | 0.25 | 0.25 | 0.25 | 8 | 0.25 | 0.25 | 1 |
| T_{1/2} (h) | 2.63 | 2.27 | | 2.18 | 1.67 | | 1.92 | 3.61 | 8.63 |
| AUCₐₗₗ | 153.28 | 1112.23 | 11459.14 | 106.70 | 524.92 | 14779.50 | 98.42 | 763.05 | 11598.60 |
| (h*ng/mL) | | | | | | | | | |

Cₘₐₓ: Peak concentration; Tₘₐₓ: Time to reach Cₘₐₓ; T_{1/2}: Half-life; AUCₐₗₗ area under de curve (all).
Noncompartmental data analysis was performed using Phoenix WinNonlin ®.
Data represents the mean values of 1-6 animals per group.

### Example 13

### Inhibition of Virus Replication of MB-905

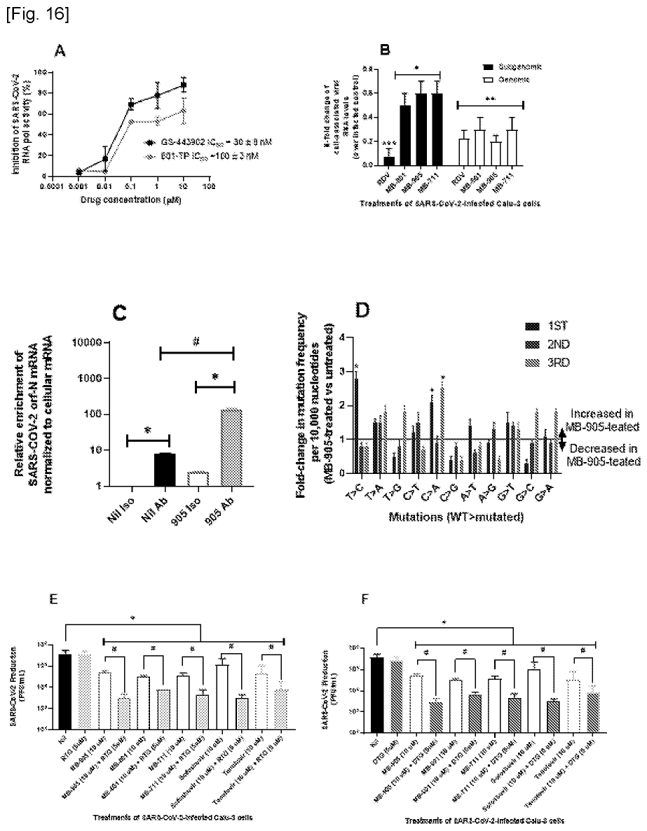

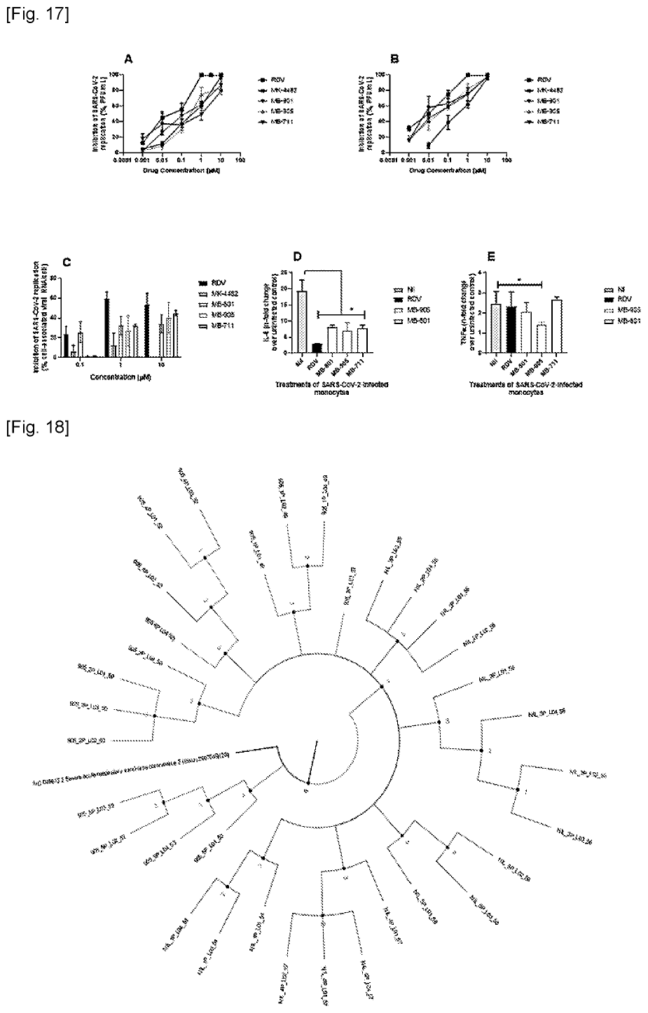

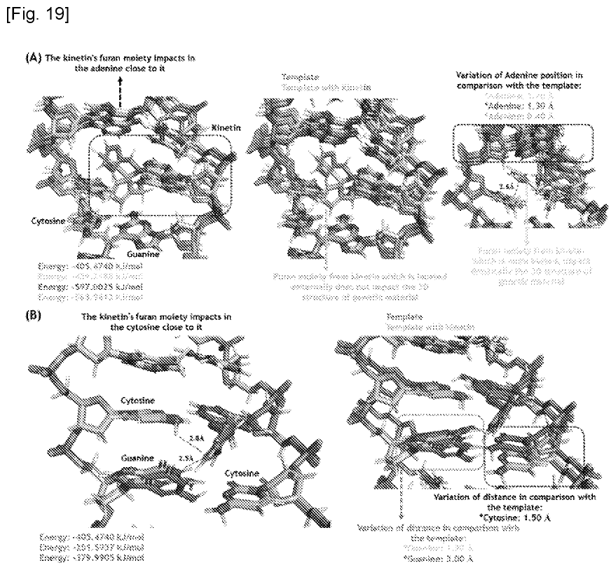

Considering that MB-905 is most likely a pro-drug of its corresponding nucleotide triphosphate, it was further tested whether inhibition of virus replication could be related to a direct action on SARS-COV-2 RNA polymerase (FIG. 16A). Kinetin riboside 5′-triphosphate inhibits the viral RNA polymerase, but with an IC50 3-fold higher when compared to the active triphosphate form of RDV (GS-443902) (FIG. 16A). RDV, MB-905 and their correspondent nucleotide triphosphates presented similar potencies, in the same order of magnitude, using cell-free (FIG. 16A) and cell-based assays (FIG. 17A, B and Table 6). Additionally, by measuring cell-associated genomic and sub-genomic viral RNA in SARS-COV-2-infected calu-3 cells, we found that compounds according to the present invention are active to impair viral RNA synthesis (FIG. 16B). Whereas RDV inhibited both the markers of replication (genomic RNA) and transcription (sub-genomic RNA) at similar magnitudes, compounds according to the present invention were more effective in reducing replication rather than transcription (FIG. 16B). Considering that MB-905 and related prodrugs deliver the riboside 5′-triphosphate in the host cell and that the latter is a substrate for SARS-COV-2 RNA polymerase, N6-furfuryladenine would be incorporated in the virus genome from MB-905-treated SARS-COV-2-infected calu-3 cells. To test this hypothesis a high affinity anti-N6-furfuryadenine IgG was used to immunoprecipitated (IP) virus RNA, follow by quantification by RT-PCR. Nil-treated cells already presented a basal level of kinetin in the viral RNA compared to control isotype immunoprecipitation (FIG. 16C), suggesting a natural occurrence of N6-furfuryladenine in the viral genome—which could be due to a natural oxidation of RNA components during sample preparation. Remarkably, viral RNA obtained from anti-N6-furfuryladenine IP of MB-905-treated SARS-COV-2-infected calu-3 cells had more than 10-fold higher levels of N6-furfuryladenine compared to isotype control and nil-treated cells (FIG. 16C). Thus, N6-furfuryladenine seems to be incorporated in the viral genome upon treatment with MB-905. We next sequenced the full-length virus genome from MB-905—and nil-treated SARS-COV-2-infected calu-3 cells and observed increased changes at the first base level, especially T(U) A and C A (FIG. 16D). The error-prone replication made the virus populations grown in the presence and absence of MB-905 phylogenetically distinguishable ([FIG. 18]). Modeling the presence of N6-furfuryladenine in a double-stranded nucleic acid highlights the N6-furfuryl moiety bumping into neighboring adenine (FIG. 19A); thus, widening the strands distance and likely impairing polymerase activity. This non-canonical base paring (FIG. 19B) could lead to an error-prone replication.

TABLE 6
In vitro Pharmacological parameters of MB-905 and related compounds
in inhibiting SARS-COV-2 virus replication in Calu-3 cells.

| | Single treatment | | | | Daily treatment | | | |
|---|---|---|---|---|---|---|---|---|
| | EC₅₀ | EC₉₀ | CC₅₀ | | EC₅₀ | EC₉₀ | CC₅₀ | |
| Compounds | [μM] | [μM] | [μM] | SI | [μM] | [μM] | [μM] | SI |
| Remdesivir | 0.15 ± | 0.4 ± | 350 ± | 2.300 | 0.01 ± | 0.2 ± | 330 ± | 3,3000 |
| | 0.03 | 0.1 | 50 | | 0.003 | 0.01 | 40 | |
| MK-4482 | 0.72 ± | 3.4 ± | 58 ± | 80 | 0.02 ± | 8.6 ± | 58 ± | 2,900 |
| | 0.12 | 0.5 | 18 | | 0.008 | 0.2 | 18 | |
| MB-905 | 0.31 ± | 2.8 ± | 620 ± | 2.000 | 0.03 ± | 2.1 ± | 538 ± | 17,933 |
| | 0.05 | 0.3 | 80 | | 0.004 | 0.3 | 64 | |
| MB-711 | 1.1 ± | 9.2 ± | 562 ± | 510 | 0.01 ± | 3.8 ± | 580 ± | 58,000 |
| | 0.03 | 0.1 | 46 | | 0.008 | 0.3 | 72 | |
| MB-801 | 0.18 ± | 7.2 ± | 550 ± | 3.055 | 0.02 ± | 3.7 ± | 530 ± | 26,500 |
| | 0.02 | 0.5 | 32 | | 0.003 | 0.2 | 65 | |

SI-Selectivity index = CC₅₀/EC₅₀

Knowing that the proof-reading mechanism in SARS-COV-2 replication complex could be actively excising the incorporated nucleotide derived from MB-905, we tested if co-inhibition of the exonuclease enhanced the in vitro antiviral activity of the compounds under investigation. Either the HIV integrase13 and HCV NS5A inhibitors 28 have been proposed to target SARS-COV-2 exonuclease. Thus, MB-905 or control RNA polymerase inhibitors were combined with suboptimal doses of dolutegravir (DTG), raltegravir (RTG), pibrantasvir (PIB), ombitasvir (OMB) or daclatasvir (Extended Table 2). We observed that combination of RNA polymerase and repurposed inhibitors of SARS-COV-2 nsp14 enhanced the potency of the former, such as MB-905 and the control antivirals (Table 7). In the case of MB-905, efficient inhibition at EC99 level was achieved when combined with the HIV integrase inhibitors (Table 6). To better demonstrate the enhancement achieved for MB-905, MB-801 or MB-711 in combination with 5 μM of DTG or RTG, results are presented as virus productive titers in untreated and treated virus-infected cells (FIG. 16E and F). Whereas compounds of the present invention inhibited virus replication in 1-log10 at 10 μM, combination with 5 μM RTG (FIG. 16E) or DTG (FIG. 16F) increased antiviral inhibition to 2-log10. Of note, RTG and DTG showed marginal effects on SARS-COV-2 replication when tested at 5 μM (FIG. 16E and F). These results on drug combination not only reinforce the characterization of MB-905 mechanism of action as an error-prone molecule, but also point out that our lead compound could be used together with repurposed drugs. Especially with DTG, which has very favorable pharmacokinetics.

TABLE 7
Pharmacological parameters for MB905 and control RdRp inhibitors alone and in combination
with SARS-COV-2 nsp14 inhibitors in Calu-3 cells

| | EC₅₀ μM | | EC₉₀ μM | | EC₉₉ μM | |
|---|---|---|---|---|---|---|
| Drug | mean | SEM | mean | SEM | mean | SEM |
| Tenofovir | 4.3 | 2.1 | ND | ND | ND | ND |
| RDV | 0.09 | 0.002 | 0.4 | 0.03 | 1.1 | 0.2 |
| Favipiravir | 7.8 | 1.2 | ND | ND | ND | ND |
| MK-4482 | 0.8 | 0.03 | 7 | 0.4 | 9 | 0.7 |
| MB905 | 0.3 | 0.02 | 8 | 1.2 | ND | ND |
| DTG | 5.3 | 1.2 | ND | ND | ND | ND |
| RTG | 4.8 | 1.4 | ND | ND | ND | ND |
| Pibrentasvir | 0.7 | 0.2 | 4.2 | 0.6 | 19 | 2 |
| Ombitasvir | 0.4 | 0.05 | 3.3 | 0.5 | 18 | 3 |
| Daclatasvir | 0.7 | 0.08 | 3.8 | 1.2 | ND | ND |
| Tenofovir + DTG (5 μM) | 0.5 | 0.03 | 7 | 1.2 | 9.8 | 0.2 |
| RDV + DTG (5 μM) | 0.09 | 0.004 | 0.4 | 0.03 | 0.9 | 0.2 |
| Favipiravir + DTG (5 μM) | 0.15 | 0.07 | 8 | 1.3 | 9.8 | 0.2 |
| MK-4482 + DTG (5 μM) | 0.03 | 0.004 | 8 | 1.2 | 9 | 0.7 |
| MB905 + DTG (5 μM) | 0.06 | 0.004 | 5 | 0.9 | 8.7 | 0.5 |
| Tenofovir + RTG (5 μM) | 0.4 | 0.02 | 8 | 1.5 | 9.5 | 0.1 |
| RDV + RTG (5 μM) | 0.08 | 0.002 | 0.5 | 0.08 | 1.2 | 0.1 |
| Favipiravir + RTG (5 μM) | 0.16 | 0.07 | 6 | 1.6 | 9.2 | 0.3 |
| MK-4482 + RTG (5 μM) | 0.01 | 0.002 | 7 | 1.4 | 9.1 | 0.5 |
| MB905 + RTG (5 μM) | 0.05 | 0.002 | 6 | 0.6 | 8.5 | 0.2 |
| Tenofovir + Pibrentasvir | 0.5 | 0.05 | 8 | 1.5 | ND | ND |
| (0.1 μM) | | | | | | |
| RDV + Pibrentasvir | 0.008 | 0.0009 | 0.07 | 0.03 | 0.3 | 0.09 |
| (0.1 μM) | | | | | | |
| Favipiravir + Pibrentasvir | 0.5 | 0.03 | 8 | 0.5 | ND | ND |
| (0.1 μM) | | | | | | |
| MK-4482 + Pibrentasvir | 5.4 | 0.3 | 7 | 0.3 | 7.8 | 0.5 |
| (0.1 μM) | | | | | | |
| MB905 + Pibrentasvir | 6.4 | 0.9 | 8 | 0.4 | ND | ND |
| (0.1 μM) | | | | | | |
| Tenofovir + Ombitasvir | 0.8 | 0.07 | 7 | 1.6 | 8.9 | 0.4 |
| (0.1 μM) | | | | | | |
| RDV + Ombitasvir | 0.08 | 0.003 | 0.01 | 0.05 | 0.5 | 0.2 |
| (0.1 μM) | | | | | | |
| Favipiravir + Ombitasvir | 0.15 | 0.04 | 8 | 0.4 | 9.5 | 0.4 |
| (0.1 μM) | | | | | | |
| MK-4482 + Ombitasvir | 0.13 | 0.02 | 4 | 0.5 | 7.8 | 0.6 |
| (0.1 μM) | | | | | | |
| MB905 + Ombitasvir | 0.3 | 0.04 | 8 | 1.3 | ND | ND |
| (0.1 μM) | | | | | | |
| Tenofovir + Dacltasvir | 0.01 | 0.004 | 6 | 1.2 | 7.5 | 0.5 |
| (0.5 μM) | | | | | | |
| RDV + Dacltasvir | 0.008 | 0.0006 | 0.1 | 0.06 | 0.5 | 0.1 |
| (0.5 μM) | | | | | | |
| Favipiravir + Dacltasvir | 0.12 | 0.05 | 8 | 0.5 | ND | ND |
| (0.5 μM) | | | | | | |
| MK-4482 + Dacltasvir | 0.02 | 0.008 | 3 | 0.4 | 8.1 | 0.3 |
| (0.5 μM) | | | | | | |
| MB905 + Dacltasvir | 0.4 | 0.03 | 4 | 0.4 | 8.8 | 0.2 |
| (0.5 μM) | | | | | | |

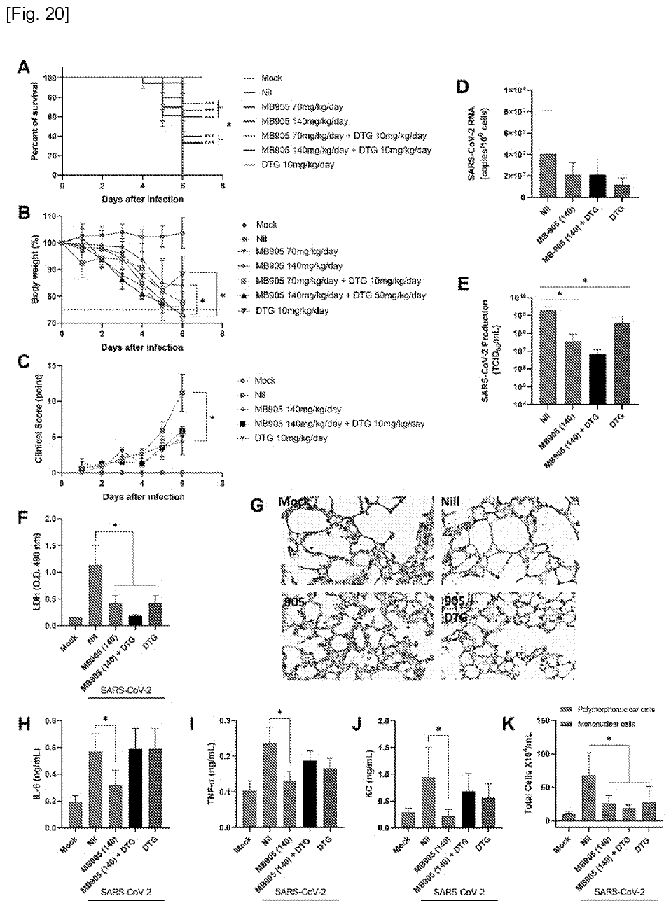

In addition, it was evaluated if MB-905 could protect transgenic mice expressing human ACE2 (K18-hACE2) from a lethal challenge (105 PFU of SARS-COV-2 VoC gamma). To better mimic a clinical situation, in which patients seek for treatment after the disease onset, oral treatments with MB-905 started 12 h after intranasal SARS-CoV-2 infection and continued thereafter, once daily. Higher survival rates were observed for MB-905 at 140 mg/kg/day, combined or not with DTG, and at 70 mg/kg/day with the HIV integrase inhibitor (FIG. 20A). Besides mortality, weight loss is the major clinical event after SARS-COV-2 infection. Under treatment with MB905 at 140 mg/kg/day alone and in combination with DTG, infected mice sustain their masses beyond the threshold of euthanasia (FIG. 20B) and improved their overall clinical scores (FIG. 20C). Although MB-905 administered with or without DTG did not alter virus RNA levels in the lungs of the infected mice (FIG. 20D), the infectious titers decreased significantly upon treatment (FIG. 20E). These results are in line with an error-prone inhibition of virus replication.

Alone or combined with DTG, MB-905 reduced lung necrosis (FIG. 20F and G). Whereas infected/untreated mice lung histology displays collapsed alveoli septum and intense hemorrhage, treated animals displayed a lung parenchyma closer to mock-infected mice (FIG. 20G). In line with in vitro results on SARS-COV-2-infected monocytes, MB-905 is also presented significant anti-inflammatory properties in vivo also. (FIGS. 20H to K). Levels of the cytokine storm markers TNF-α (FIG. 20H), IL-6 (FIG. 201) and KC (FIG. 20J), and inflammatory cells counts (FIG. 20K) were reduced in the bronchioalveolar lavage (BAL) of SARS-COV-2-infeced mice treated with MB-905, in comparison with the untreated animals.

### Example 14

### In Vivo Inhibition of Betacoronavirus Replication by MB-905

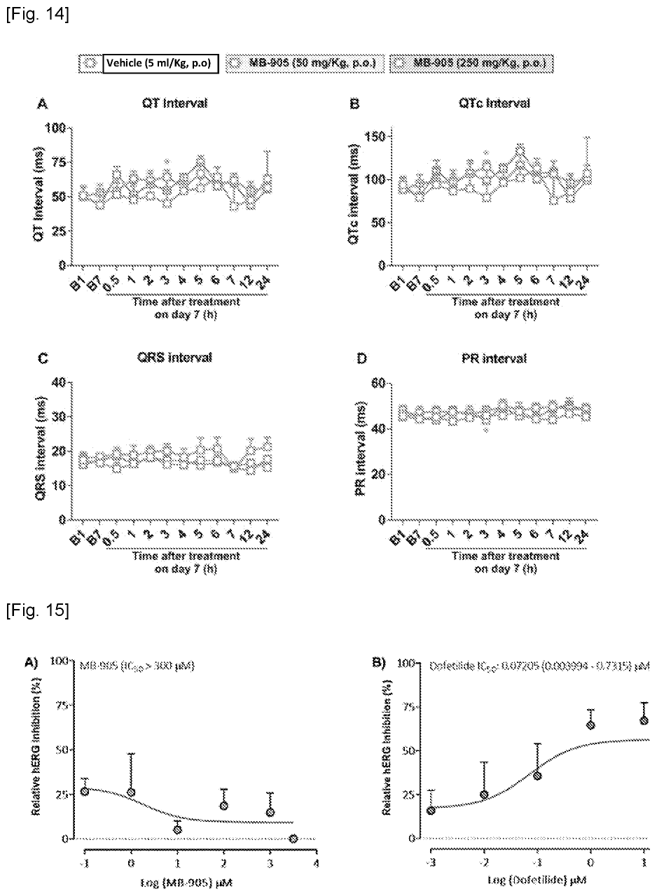

Enzymatic machinery to sustain betacoronavirus replication is very conserved, with homologies between SARS-COV-2 and murine hepatitis virus (MHV) above 70% for nsp12 and nsp14 (FIG. 14A). In fact, the active site for both these critical enzymes during RNA synthesis is identical (FIG. 14A), based on translated proteins from genbank (nsp12, YP_009924352.1 vs YP_009725307.1; nsp14, YP_009924354.1 vs YP_009725309.1). Considering the restrictions to access animal biosafety level 3 (ABSL-3) facilities internationally, the prototypic betacoronavirus MHV is an alternative for in vivo testing at ABSL-2. This is especially consistent for drugs that target the replication machinery. Upon intranasal inoculation, we observed 50% mortality (FIG. 14B). MB-905 alone reduced the mice mortality by half, whereas daclatasvir (DAC)—which favors SARS-COV-2 RNA to unfold secondary structures and prevent nsp12 activity—completely prevented mortality (FIG. 14B). Although drugs did not allow complete weight recovery (FIG. 14C), less inflammatory cells were detected in the respiratory tract of the infected mice (FIG. 14D). This is consistent with example 5, when MBs ability to carly impair SARS-COV-2 replication led to anti-inflammatory activity.

### Example 15

### Genotoxicity Evaluation of MB-905—AMES Test

Genotoxicity tests were developed to detect substances with the potential to induce damage to genetic material and are recommended by regulatory agencies worldwide as part of the safety assessment of chemicals. These tests identify risks related to DNA damage. Substances that test positive in these tests that detect genetic modifications are potentially carcinogenic and/or mutagenic to humans. Thus, the bacterial mutagenicity test is widely used as an initial screening to assess possible genotoxic activity, in particular, for point mutation-inducing activity. The reverse bacterial mutation assay was performed followed the recommendations of OECD guide 471—Guideline for Testing of Chemicals. Method 471 “Bacterial Reverse Mutation Test” (Adopted: 26 June 2020). The preliminary test with the strain TA 100, in the absence or presence of metabolic activation (S9) was conducted with the goal of selecting adequate concentrations of the MB-905 for the definitive test. The results are showing in table 8.

TABLE 8
Evaluation of possible Mutagenicity effect of MB-905 tested in the Ames tester
strains TA 97a, TA 98, TA100, TA102 and TA 1535. The test was performed in
the absence and presence of the metabolic activation system (8% of S9 in the
mixture with required co-factors).

| | Concentration | TA 97a | | TA 98 | | TA 100 | | TA 102 | | TA 1535 | |
|---|---|---|---|---|---|---|---|---|---|---|---|
| Treatments | (μg/mL) | −S9 | +S9 | −S9 | +S9 | −S9 | +S9 | −S9 | +S9 | −S9 | +S9 |
| MB-905 | 8 | − | − | − | − | − | − | − | − | − | − |
| | 40 | − | − | − | − | − | − | − | − | − | − |
| | 200 | − | − | − | − | − | − | − | − | − | − |
| | 1,000 | − | − | − | − | − | − | − | − | − | − |
| | 5,000 | − | − | − | − | − | − | − | − | − | − |
| Positive | # | + | + | + | + | + | + | + | + | + | + |
| Control | | | | | | | | | | | |

(−S9) = absence of the metabolic activation system; (+S9) = presence of the metabolic activation system (−) = negative; (+) = positive. # Positive controls = 4-nitroquinoline-N-oxide (4NQO) 0.5 μg/plate: TA97a, TA98 and TA102 (-S9); sodium azide (AZS) 1.5 μg/plate: TA100 and TA 1535 (−S9); 2-aminofluorene (2-AF) 50 μg/plate: TA97a, TA98 and TA100 (+S9); 2-aminoanthracene (2-AA): 2.5 and 5 μg/plate: TA 1535 and TA102, respectively (+S9).

The results demonstrate that no mutagenic activity was observed to MB-905 in the reverse mutation test in Salmonella typhimirium bacteria, both in the absence and in the presence of metabolic activation (S9), in the TA 97a, TA 98, TA 100, TA 102 and TA 1535.

### Example 16

### Genotoxicity Evaluation of MB-905—Micronucleous Test

The micronucleus was performed in mouse bone marrow in accordance to the OECD guideline 474 and conducted in compliance with the GLP principles. Male and female Swiss mice (5-10 weeks) were divided into 5 experimental groups and were treated orally with vehicle (5% Tween+95% PEG E 400), three different doses of MB-905 (32, 125 or 500 mg/kg) for three consecutive days or with cyclophosphamide (25 mg/kg, i.p.) for 2 consecutive days. Bone marrow cells were collected and processed according to a methodology described by Schmid (1975). The ratio of polychromatic to normochromatic erythrocytes and the count of micronuclei were determined. This assay was conducted in compliance with the GLP principles. The results are showing in table 9.

TABLE 9
Incidence of micronucleated polychromatic erythrocytes (MNPCE)
and the ratio of polychromatic erythrocytes (PCE) to normochromatic
erythrocytes in mice treated with MB-905.

| | | | | |
|---|---|---|---|---|
| | | | MNPCE/4,000 | Ratio |
| | Dose | | PCE | PCE/NCE |
| Group | (mg/Kg) | Route | (Mean ± S.D.) | (Mean ± S.D.) |

| | | | |
|---|---|---|---|
| Negative Control | 0 | p.o. | 10.10 ± 4.89 |
| (Water) | | | |
| Test Item | 32 | p.o. | 8.50 ± 4.50 |
| (MB-905) | | | |
| Test Item | 125 | p.o. | 8.50 ± 3.81 |
| (MB-905) | | | |
| Test Item | 500 | p.o. | 9.00 ± 2.45 |
| (MB-905) | | | |
| Positive Control | 25 | i.p. | 16.20 ± 6.03* |
| (Cyclophosphamide) | | | |

p.o. = per os; i.p. = intraperitoneal; PCE = polychromatic erythrocytes; NCE = nor-mochromatic erythrocytes; MNPCE = micronucleated polychromatic erythrocytes; S.D. = standard deviation.
*Significant difference from negative control by Kruskal-Wallis test: *p < 0.05.

It can be concluded that, under the conditions evaluated and, in the doses tested, MB-905 did not promote an increase in the number of micronucleated PCEs in mice and therefore has no genotoxic action.

### Example 17

### Maximum Tolerated Dose and Dose Selection in Rats

The present assay was designed to investigate the safety and tolerability of MB-905. For the MTD assay (OECD 425), mice (3 animals of each sex/group) were divided into five experimental groups and the up and down procedure was applied to which 175 mg/kg was used as the first dose. Then, since the MTD assay pointed out 550 mg/kg as the recommended dose for repeated exposure, tolerability of MB-905 was assessed by submitting two experimental groups (5 of each sex/group) to an oral treatment with the vehicle (group 1) or with MB-905 (550 mg/kg) for 7 consecutive days. Mortality, morbidity, body weight, food consumption, general and detailed clinical signs of toxicity were evaluated. At necropsy, vital (brain, heart, liver, spleen, kidneys and adrenal glands) and reproductive organs (ovaries, testicles and epididymis) were carefully removed, weighed and stored for further analysis (if necessary).

Morbidity and mortality—The oral treatment of animals with MB-905 with the doses of 175, 550 and 850 mg/kg, by oral route did not result in any signs indicative of toxicity of the animals during the whole treatment period. However, mice treated orally with 1,150 mg/kg of MB-905 resulted in death within 4 hours after compound administration.

General and detailed clinical signs: The detailed clinical signs were performed once before the beginning of the treatments to verify the health status of the animals, and once a week thereafter. General clinical signs were evaluated every hour, up to the fourth hour after treatment, and then daily. Animals treated orally with MB-905 at different dose levels (175, 550 and 850 mg/kg) did not result in any observable clinical signs indicating toxicity throughout the experimental period. Mice that survived after treatment with 1,150 mg/kg of MB-905 exhibited piloerection, reduced touch response, prostration, loss of grasping strength, decrease in body temperature and death of the animals, within 4 hours after oral administration.

Body weight change and food consumption: Body weight and food consumption was measured once before the start of treatments (baseline) and then once a week. For both parameters it was not observed any significant change related to the single treatment with MB-905 (175, 550 or 850 mg/kg) at the end of the experimental protocol.

Organs weight: After the necropsy procedure, the weight (g) of the principal organs (adrenal glands, spleen, brain, heart, kidney, thymus, liver, testis, epididymis and ovary) was measured for each animal in all experimental groups. The results did not show any changes related to the single oral treatment with MB-905 (175, 550 or 850 mg/kg).

The NOAEL (Not Observable Adverse Effect Level) for oral administration of MB-905 to mice was estimated to be 550 mg/kg.

### Example 18

### Repeated Dose 28-Day Toxicity Study of MB-905 in Mice With Toxicokinetics

The purpose of this study was to assess the potential toxicity after treatment with MB-905 administered by oral route for 28 days in mice. Male and female CD1 mice (6-8 weeks, 10 mice/group/sex) were treated orally with vehicle (45% polyethylene glycol 400—PEG 400, 30% propylene glycol, 20% filtered water and 5% ethanol) or with different doses of MB-905 (10 mg/kg, 80 mg/kg or 250 mg/kg), once daily for 28 days (main animals). Additionally, recovery groups (5 males and 5 females) were established to which the same treatment regimen was applied but animals (Vehicle or MB-905 250 mg/kg) remained untreated for another 14 days in order to observe persistence, reversibility or delayed occurrence of toxic effects related to the administration of the Test Item. These experiments were conducted in compliance with the GLP principles. Morbidity, mortality, body weight in male (FIG. 10A) and female (FIG. 10B), food consumption as well as general and detailed clinical signs were evaluated. In the last week, urine samples from all animals were collected for analysis. At necropsy, blood samples were collected for hematological, biochemical and coagulation analyses, followed by tissue and organ collection for macroscopic and histopathological analyses.

Clinical chemistry and hematology: Main groups: MB-905 led to slight changes in biochemical and hematological parameters in male (table 10 and 12) and female (table 11 and 13) that were reversed in the animals of the Recovery groups.

TABLE 10
Analysis of hematological parameters of male mice after 28 days of daily treatment with MB-905.
MALE

| | | WBC | RBC | | | | | | PLT | W-S | W-M | W-L |
|---|---|---|---|---|---|---|---|---|---|---|---|---|
| | | (×10³/ | (×10⁶/ | HGB | HCT | MCV | MCH | MCHC | (×10³/ | CR | CR | CR |
| GROUP | | μL) | μL) | (g/dL) | (%) | (fL) | (pg) | (g/dL) | μL) | (%) | (%) | (%) |
| VEHICLE | Mean | 3.20 | 7.50 | 12.64 | 38.83 | 51.84 | 16.89 | 32.54 | 1056.00 | 62.10 | 6.66 | 30.69 |
| (Vehicle) | DP^{&} | 1.23 | 0.30 | 0.60 | 1.63 | 1.32 | 0.41 | 0.37 | 68.18 | 9.30 | 2.96 | 9.29 |
| | N | 9 | 9 | 9 | 9 | 9 | 9 | 9 | 9 | 9 | 9 | 9 |
| DOSE 1 | Mean | 1.61 | 7.30 | 12.51 | 38.33 | 52.46 | 17.12 | 32.69 | 971.90 | 51.88 * | 5.41 | 40.81 * |
| (10 mg/kg) | DP^{&} | 0.48 | 0.43 | 0.80 | 2.81 | 1.29 | 0.38 | 0.57 | 172.83 | 5.09 | 2.21 | 5.15 |
| | N | 10 | 10 | 10 | 10 | 10 | 10 | 10 | 10 | 10 | 10 | 10 |
| DOSE 2 | Mean | 4.70 | 7.28 | 12.64 | 38.59 | 53.00 | 17.34 | 32.76 | 1034.44 | 79.52 * | 3.36 * | 16.37 * |
| (80 mg/kg) | DP^{&} | 2.01 | 0.43 | 0.72 | 1.82 | 1.32 | 0.54 | 0.56 | 230.86 | 3.79 | 0.75 | 3.39 |
| | N | 9 | 9 | 9 | 9 | 9 | 9 | 9 | 9 | 9 | 9 | 9 |
| DOSE 3 | Mean | 4.11 | 7.79 | 13.26 | 40.41 | 51.91 | 17.03 | 32.80 | 1056.33 | 74.40* | 4.17 | 20.89 * |
| (250 mg/kg) | DP^{&} | 1.81 | 0.42 | 0.75 | 2.05 | 1.34 | 0.58 | 0.52 | 149.37 | 6.22 | 1.11 | 6.07 |
| | N | 9 | 9 | 9 | 9 | 9 | 9 | 9 | 9 | 9 | 9 | 9 |

^{&}Standard deviation; N-number of animals; *Differs significantly in relation to the Vehicle group; WBC-Total Cells; RBC-Red blood cells; HGB-Hemoglobin; HCT-Hematocrit; MCV-Corpuscular Volume; MCH-Mean corpuscular hemoglobin; MCHC-Mean corpuscular hemoglobin concentration; PLT-Platelet count; W-SCR-Small leukocyte (lymphocyte) index; W-MCR-Mean leukocyte (monocyte) index; W-LCR-Large leukocyte (neutrophil) index.

TABLE 11
Analysis of hematological parameters of female mice after 28 days of daily treatment with MB-905.
FEMALE

| | | WBC | RBC | | | | | | PLT | W-S | W-M | W-L |
|---|---|---|---|---|---|---|---|---|---|---|---|---|
| | | (×10³/ | (×10⁶/ | HGB | HCT | MCV | MCH | MCHC | (×10³/ | CR | CR | CR |
| GROUP | | μL) | μL) | (g/dL) | (%) | (fL) | (pg) | (g/dL) | μL) | (%) | (%) | (%) |
| VEHICLE | Mean | 2.25 | 7.76 | 12.18 | 40.27 | 51.97 | 17.30 | 33.29 | 881.80 | 60.50 | 4.25 | 26.25 |
| (Vehicle) | DP^{&} | 1.10 | 0.41 | 3.82 | 1.52 | 1.19 | 0.57 | 0.44 | 268.40 | 13.51 | 4.62 | 5.91 |
| | N | 10 | 10 | 10 | 10 | 10 | 10 | 10 | 10 | 10 | 10 | 10 |
| DOSE 1 | Mean | 1.70 | 7.76 | 13.58 | 40.60 | 52.32 | 17.51 | 31.74 | 1073.22 | 57.60 | 5.03 | 36.41 |
| (10 mg/kg) | DP^{&} | 0.81 | 0.35 | 0.31 | 1.27 | 1.16 | 0.51 | 5.17 | 161.81 | 12.27 | 2.95 | 10.73 |
| | N | 9 | 9 | 9 | 9 | 9 | 9 | 9 | 9 | 9 | 9 | 9 |
| DOSE 2 | Mean | 3.59 | 7.61 | 13.31 | 39.78 | 52.32 | 17.50 | 33.47 | 918.00 | 74.94 * | 2.77 | 21.77 |
| (80 mg/kg) | DP^{&} | 1.26 | 0.32 | 0.31 | 1.60 | 0.86 | 0.47 | 0.67 | 113.59 | 8.03 | 1.08 | 7.63 |
| | N | 9 | 9 | 9 | 9 | 9 | 9 | 9 | 9 | 9 | 9 | 9 |
| DOSE 3 | Mean | 4.88* | 7.61 | 13.27 | 40.70 | 52.80 | 17.46 | 33.03 | 780.33 | 73.06 | 3.10 | 22.91 |
| (250 mg/kg) | DP^{&} | 1.81 | 0.33 | 0.60 | 1.82 | 1.14 | 0.49 | 0.44 | 293.49 | 12.71 | 2.06 | 10.31 |
| | IN | 9 | 9 | 9 | 9 | 9 | 9 | 9 | 9 | 9 | 9 | 9 |

^{&}Standard deviation; N-number of animals; * Differs significantly in relation to the Vehicle group; WBC-Total Cells; RBC-Red blood cells; HGB-Hemoglobin; HCT-Hematocrit; MCV-Corpuscular Volume; MCH-Mean corpuscular hemoglobin; MCHC-Mean corpuscular hemoglobin concentration; PLT-Platelet count; W-SCR-Small leukocyte (lymphocyte) index; W-MCR-Mean leukocyte (monocyte) index; W-LCR-Large leukocyte (neutrophil) index.

TABLE 12
Analysis of biochemical parameters of male mice after 28 days of daily treatment with MB-905.
MALE

| | | ALT | GGT | TRI | PT | CRE | AST | CA | GLUCOSE |
|---|---|---|---|---|---|---|---|---|---|
| GROUP | | (U/L) | (U/L) | (mg/dL) | (g/dL) | (mg/dL) | (U/L) | (mg/dL) | (mg/dL) |
| VEHICLE | Mean | 55.80 | 0.00 | 113.90 | 5.78 | 0.27 | 109.34 | 10.64 | 139.68 |
| (Vehicle) | DP^{&} | 11.38 | 0.00 | 27.045 | 0.80 | 0.07 | 30.61 | 1.58 | 39.19 |
| | N | 8 | 6 | 10 | 10 | 7 | 8 | 6 | 10 |
| DOSE 1 | Mean | 43.99 | 2.33 | 94.50 | 5.56 | 0.37 | 105.30 | 9.91 | 127.41 |
| (10 mg/kg) | DP^{&} | 32.72 | 5.72 | 37.51 | 0.81 | 0.13 | 33.07 | 1.07 | 29.80 |
| | N | 8 | 6 | 10 | 9 | 6 | 7 | 6 | 10 |
| DOSE 2 | Mean | 69.54 | 0.00 | 116.7 | 5.74 | 0.28 | 134.48 | 9.76 | 129.24 |
| (80 mg/kg) | DP^{&} | 53.28 | 0.00 | 8 | 0.35 | 0.10 | 38.95 | 0.93 | 13.84 |
| | N | 9 | 8 | 35.979 | 9 | 8 | 9 | 5 | 9 |
| DOSE 3 | Mean | 57.17 | 0.50 | 84.90 | 5.22 | 0.31 | 124.78 | 10.38 | 105.23 |
| (250 mg/kg) | DP^{&} | 26.68 | 1.07 | 46.64 | 0.82 | 0.13 | 52.36 | 0.72 | 35.15 |
| | N | 9 | 8 | 10 | 10 | 8 | 8 | 8 | 10 |

MALES

| | | BT | CT | FA | P | UREIA | ALB |
|---|---|---|---|---|---|---|---|
| GROUP | | (mg/dL) | (mg/dL) | (U/L) | (mg/dL) | (mg/dL) | (mg/dL) |
| VEHICLE | Mean | 0.54 | 146.63 | 94.83 | 5.11 | 67.25 | 2.92 |
| (Vehicle) | DP^{&} | 0.51 | 28.73 | 26.52 | 0.79 | 10.04 | 0.56 |
| | N | 9 | 8 | 6 | 8 | 6 | 6 |
| DOSE 1 | Mean | 0.67 | 137.00 | 103.67 | 4.54 | 60.48 | 2.76 |
| (10 mg/kg) | DP^{&} | 0.63 | 34.62 | 33.90 | 0.47 | 9.17 | 0.36 |
| | N | 9 | 10 | 6 | 8 | 6 | 6 |
| DOSE 2 | Mean | 0.25 | 154.56 | 69.67 | 5.07 | 30.02 * | 2.72 |
| (80 mg/kg) | DP^{&} | 0.24 | 16.02 | 21.03 | 0.53 | 1.51 | 0.22 |
| | N | 9 | 9 | 6 | 9 | 5 | 5 |
| DOSE 3 | Mean | 0.43 | 183.56 | 85.88 | 4.22 | 74.16 | 2.82 |
| (250 mg/kg) | DP^{&} | 0.30 | 27.75 | 21.58 | 0.90 | 6.64 | 0.31 |
| | N | 10 | 9 | 8 | 9 | 8 | 8 |

Standard deviation; N-number of animals; * Differs significantly in relation to the Vehicle group; ALT-Alanine aminotransferase; GGT-Gamma-glutamyltransferase; TRI-Triglycerides; EN-Total protein; CRE-Creatinine; ALB-Albumin; AST-Aspartate Aminotransferase, BT-Total Bilirubin; TC-Total cholesterol; FA-Alkaline phosphatase; P-Phosphorus; Ca-Calcium.

TABLE 13
Analysis of biochemical parameters of male mice after 28 days of daily treatment with MB-905.
FEMALE

| | | ALT | GGT | TRI | PT | CRE | AST | CA | GLUCOSE |
|---|---|---|---|---|---|---|---|---|---|
| GROUP | | (U/L) | (U/L) | (mg/dL) | (g/dL) | (mg/dL) | (U/L) | (mg/dL) | (mg/dL) |
| VEHICLE | Mean | 36.11 | 0.00 | 63.88 | 5.17 | 0.29 | 76.25 | 9.80 | 127.66 |
| (Vehicle) | DP^{&} | 12.47 | 0.00 | 13.29 | 0.51 | 0.02 | 58.47 | 0.79 | 28.58 |
| | N | 8 | 5 | 8 | 8 | 5 | 8 | 5 | 7 |
| DOSE 1 | Mean | 43.72 | 0.00 | 62.00 | 5.84 | 0.29 | 91.88 | 9.77 | 118.56 |
| (10 mg/kg) | DP^{&} | 11.41 | 0.00 | 14.31 | 0.76 | 0.03 | 26.79 | 1.07 | 31.01 |
| | N | 5 | 3 | 7 | 7 | 4 | 4 | 4 | 7 |
| DOSE 2 | Mean | 52.50 | 0.00 | 92.90 | 5.99 | 0.25 | 119.73 | 8.34 | 138.04 |
| (80 mg/kg) | DP^{&} | 20.98 | 0.00 | 50.88 | 0.88 | 0.07 | 20.34 | 2.99 | 34.71 |
| | N | 6 | 6 | 10 | 10 | 6 | 6 | 5 | 10 |
| DOSE 3 | Mean | 44.84 | 0.20 | 108.33 | 6.79 | 0.30 | 127.67 | 10.26 | 135.84 |
| (250 mg/kg) | DP^{&} | 5.72 | 0.45 | 19.65 | 1.73 | 0.06 | 28.51 | 2.64 | 22.65 |
| | N | 7 | 5 | 9 | 9 | 5 | 5 | 4 | 9 |

FEMALE

| | | BT | CT | FA | P | UREIA | ALB |
|---|---|---|---|---|---|---|---|
| GROUP | | (mg/dL) | (mg/dL) | (U/L) | (mg/dL) | (mg/dL) | (g/dL) |
| VEHICLE | Mean | 0.55 | 101.71 | 104.40 | 5.40 | 54.20 | 2.87 |
| (Vehicle) | DP^{&} | 0.18 | 18.55 | 24.10 | 1.69 | 5.37 | 0.37 |
| | N | 7 | 7 | 5 | 8 | 5 | 5 |
| DOSE 1 | Mean | 0.39 | 98.17 | 108.75 | 4.69 | 54.93 | 2.67 |
| (10 mg/kg) | DP^{&} | 0.26 | 46.39 | 39.49 | 2.22 | 7.64 | 0.34 |
| | N | 7 | 6 | 4 | 6 | 4 | 4 |
| DOSE 2 | Mean | 0.41 | 119.86 | 77.20 | 5.47 | 44.12 | 2.91 |
| (80 mg/kg) | DP^{&} | 0.46 | 22.00 | 45.49 | 0.88 | 10.83 | 0.03 |
| | N | 8 | 7 | 5 | 7 | 5 | 5 |
| DOSE 3 | Mean | 0.34 | 126.67 | 80.40 | 5.44 | 73.50 | 3.29 |
| (250 mg/kg) | DP^{&} | 0.22 | 36.40 | 50.87 | 1.59 | 27.27 | 1.19 |
| | N | 9 | 9 | 5 | 7 | 4 | 4 |

Standard deviation; N-number of animals; * Differs significantly in relation to the Vehicle group; ALT-Alanine aminotransferase; GGT-Gamma-glutamyltransferase; TRI-Triglycerides; EN-Total protein; CRE-Creatinine; ALB-Albumin; AST-Aspartate Aminotransferase, BT-Total Bilirubin; TC-Total cholesterol; FA-Alkaline phosphatase; P-Phosphorus; Ca-Calcium.

Urinalysis: No significant changes in urine parameters (volume, specific gravity, pH, protein) was observed in main groups.

Ophthalmology: No changes were observed in the ophthalmological health of either sex in the highest dose (250 mg/kg).

Macroscopy: Macroscopic evaluations performed during the necropsy procedure did not reveal significant changes related to the treatment.

Histopathology: Histopathological evaluations revealed effects on the kidneys related to treatment with MB-905 at a dose of 250 mg/kg, in both sexes. The changes on the kidneys were characterized by areas of basophilic proximal tubules and tubular dilatation, which were not observed in 10 mg/kg and 80 mg/kg doses, as well as in the recovery group.

The NOAEL (Not Observable Adverse Effect Level) for oral administration of MB-905 to mice was estimated to be 80 mg/kg.

Toxicokinetics parameters: Through the AUCall and Cmax data from day 0 and day 28 of the study, indicate that no there was an accumulation of the MB-905, in both males and females, indicating that possible toxic effects of the compound could be quickly recovery by treatment interruption (Table 14).

TABLE 14
Toxicokinetics parameters of male and female mice after 28 days of daily treatment with MB-905

| | Day 0 | | | | Day 28 | | | |
|---|---|---|---|---|---|---|---|---|
| | Cₘₐₓ | Tₘₐₓ | T_{1/2} | AUCₐₗₗ | Cₘₐₓ | Tₘₐₓ | T_{1/2} | AUCₐₗₗ |
| | (ng/mL) | (h) | (h) | (h*ng/mL) | (ng/mL) | (h) | (h) | (h*ng/mL) |
| FEMALE | | | | | | | | |
| 10 (mg/kg) | 287.54 | 0.25 | ND | 422.23 | 95.05 | 0.25 | ND | 56.64 |
| 80 (mg/kg) | 747.41 | 0.25 | 25.91 | 5005.59 | 464.29 | 0.25 | 1.09 | 1588.47 |
| 250 (mg/kg) | 898.42 | 0.25 | 3.08 | 4914.08 | 715.78 | 0.25 | 2.59 | 2080.90 |
| MALE | | | | | | | | |
| 10 (mg/kg) | 99.02 | 0.25 | ND | 91.05 | 144.33 | 0.25 | ND | 121.26 |
| 80 (mg/kg) | 597.59 | 0.25 | 1.21 | 1019.27 | 749.76 | 0.25 | ND | 1171.80 |
| 250 (mg/kg) | 792.90 | 1 | ND | 4475.43 | 588.13 | 0.25 | 1.53 | 1131.36 |

Cₘₐₓ: Peak concentration; Tₘₐₓ: Time to reach Cₘₐₓ; T_{1/2}: half-life; AUCₗₐₛₜ: Area under de curve (last); AUCₐₗₗ area under de curve (all).

### Example 19

### Evaluation of Cardiovascular Safety Pharmacology of MB-905 by Telemetry

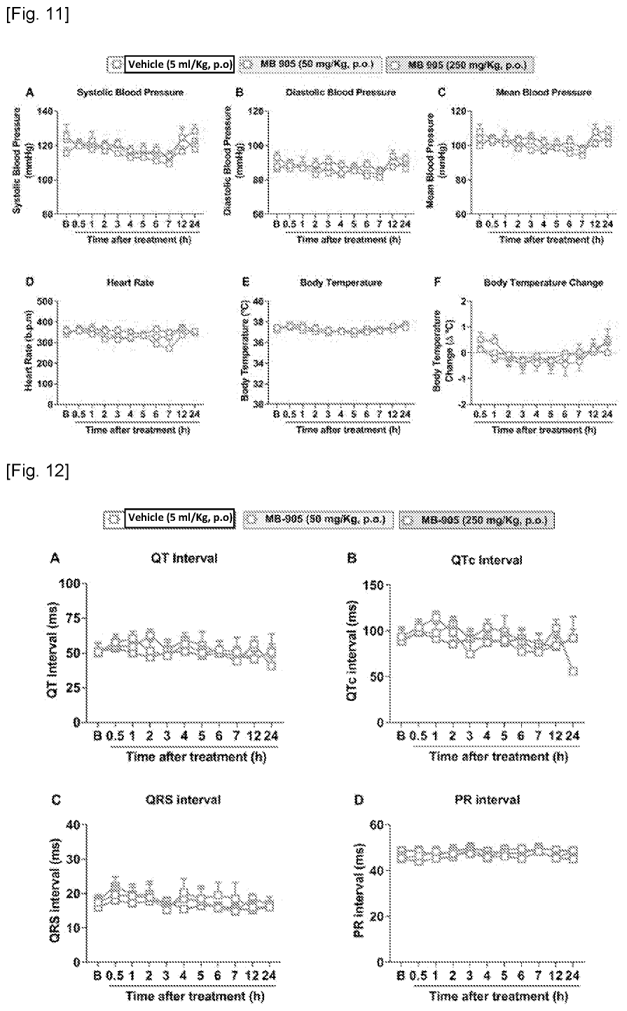

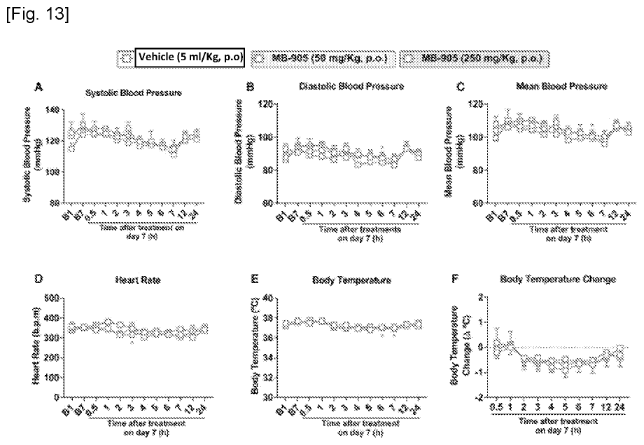

Conscious and freely moving male Sprague-Dawley rats (9-12 weeks), previously submitted to surgery for placement of the DSITM PhysioTel hardware system implant in the abdominal aorta, were treated orally with Vehicle (5 ml/kg) or MB-905 (50 or 250 mg/kg) once a day for 7 consecutive days. Cardiovascular parameters such as systolic blood pressure, diastolic blood pressure, heart rate and electrocardiogram were evaluated before treatments (baseline) and at 0.5, 1, 2, 3, 4, 5, 6, 7, 12 and 24 hours after the treatments on days 1 and 7. Results indicates that single or repeated administration of MB-905 (50 or 250 mg/Kg) did not induces any changes in the cardiovascular hemodynamic parameters, such as systolic blood pressure, diastolic blood pressure, mean blood pressure and heart rate (FIGS. 11 and 13), when compared to vehicle treated animals. Also, when compared to vehicle group, MB-905 (50 or 250 mg/Kg) did not induce any change in the electrocardiogram parameters (QT interval, QTc interval, QRS interval and PR interval), when single ([FIG. 12]) or repeated ([FIG. 14]) administrated. Considering the results obtained, the MB-905 doses tested and the conditions in which the experiments were carried out, it is possible to conclude that MB-905 presents a safe profile regarding the cardiovascular system when evaluated in freely moving rats telemetry assay.

Example 20

Inhibition of Voltage-Dependent Potassium Channels of the hERG Type (Human Ether-a-go-go Related)

The voltage-dependent potassium channels of the hERG type (human ether-a-go-go related) are essential for normal electrical activity in the heart. hERG channel dysfunction can cause long QT syndrome (LQTS), characterized by delayed repolarization and prolongation of the QT interval of the cardiac cell's action potential, which increases the risk of ventricular arrhythmias and sudden death. Thus, compounds that act in this channel and that has potential to cause long QT syndrome have been eliminated early in the process of non-clinical development in safety tests.

Studies that aim to evaluate the inhibition of the potassium channel hERG, are traditionally carried out through electrophysiology tests, using the patch clamp technique, which is considered the gold standard for ion channel studies; however, other methodologies have been developed in order to assess the influx of ions through ion channels transfected into immortalized cells. One of these methodologies consists in using the commercial kit called FLIPRR Potassium Assay, which is increasingly used for the evaluation and rapid and robust screening of compounds on ion channels, such as the hERG channel. The method used in this test was based on the permeability of the hERG potassium channels to thallium, a component present in the commercial FLIPRR Potassium Assay kit. When the hERG potassium channels are opened by a stimulus, the influx of thallium from the external environment is detected by a highly sensitive indicator dye. The fluorogenic signal quantitatively reflects the activity of hERG ion channels that are permeate to thallium. The results of validating the methodology using the FLIPRR Potassium Assay, when compared to electro-physiology studies, demonstrated that both methods produce equivalent results on the hERG channel. Therefore, to assess the interaction with hERG, the commercial kit FLIPRR Potassium Assay (Molecular Devices) was used and the test was performed according to the manufacturer's specifications.

The recombinant HEK-293 cell line for the expression of the human hERG gene Kv11.1) was acquired from the company BPS Bioscience. For use in the present study, the cells were thawed and cultured according to the supplier's specifications: hERG (Kv11.1)—HEK-293 Recombinant Cell line Cat #: 60619 product sheets. The cells were kept in bottles containing supplemented culture medium, in a CO₂ incubator, at 37° C. with 5% and 0.2% CO₂, until the time of the tests. For this, after thawing the HEK-293 cells transfected with human hERG, they were plated at a density of 4×104 cells per well in a black 96-well, flat, transparent bottom plate. After the confluence of the cells, the plate culture medium was aspirated and replaced with 50 μL of HBSS calcium and magnesium free. Then, the cells were incubated with 50 μL of the fluorescent probe present in the commercial kit, containing probenecid in the final concentration of 2.5 mM. After 1 hour of incubation at room temperature and in the dark, 25 μL of treatments with ST-080 were added to the wells, and the plate was incubated again for 30 minutes. The previously optimized stimulus buffer (50 μL of 1 mM thallium+10 mM potassium) was added to each column through automated pipetting present in the FlexStation 3 equipment. The signal was acquired at intervals of 1.52 seconds for approximately 140 seconds per column. The data were obtained using the SoftMaxRPro Software, at an excitation wavelength of 485 nm and an emission wavelength of 538 nm. Data analysis was performed using SoftMax Pro Software and GraphPad PrismR 8. The results were expressed as percentage of inhibition of the hERG channel and the mean inhibitory concentration (IC₅₀) and the respective 95% confidence intervals were calculated using linear regression.

As seen in [FIG. 15] the compound MB-905 (A) incubated in cells even at very higher concentration (up to 300 μM) that largely exceed those observed in rodent plasma caused a very low inhibition (about 20%) potassium permeation through the hERG channel. On the other hand, dofetilide, a reported selective inhibitor of hERG channel, produced a concentration-dependent inhibition of hERG channels. The estimated mean IC₅₀ concentration of dofetilide (FIG. 15B) of the hERG channel activity (IC50) was 0.012 μM.

A person having skills in the concerned art, by way of the explanations and examples comprised herein will promptly appreciate the advantages of the invention and will be able to propose equivalent embodiments of the invention without departing from the scope of the attached inventions.

1. Antiviral compound that inhibits viral RNA synthesis for the prophylactic, therapeutic or mitigative treatment against coronavirus, in especial SARS-COV-2, and for the treatment of patients with COVID-19, and individuals potentially exposed to or at risk of exposure to coronavirus, in especial SARS-CoV-2 characterized by the fact that it is selected from the group of kinetin (MB-905), kinetin riboside (MB-801), kinetin riboside monophosphoramidate (MB-711), zeatin (MB-907), and zeatin riboside (MB-804) or their derivatives, salts, solvates or prodrugs.

2. Compound, according to claim 1, characterized by the fact that it is selected from active monophosphate and diphosphates and triphosphates of kinetin (MB-905), kinetin riboside (MB-801), kinetin riboside monophosphoramidate (MB-711), zeatin (MB-907), and zeatin riboside (MB-804) or their derivatives, salts, solvates or prodrugs.

3. Compound, according to claim 1, characterized by the fact that the compound is kinetin-ribose 5′-triphosphate (MB-717).

4. Antiviral pharmaceutical composition, characterized by the fact that it comprises (i) an effective amount of one or more antiviral compounds, as defined in claim 1; and (ii) pharmaceutically acceptable excipients compatible with the active ingredients; for the prophylactic, curative (therapeutic) or mitigative treatment of against coronavirus, in especial SARS-COV-2 and for the treatment of patients with COVID-19, and individuals potentially exposed to or at risk of exposure to SARS-COV-2.

5. Antiviral pharmaceutical composition, according to claim 4, characterized by the fact that it further comprises (iii) a synergized amount of raltegravir, dolutegravir and their analogs.

6. Antiviral pharmaceutical composition, according to claim 4, characterized by the fact that it contains from 1 to 3,000 mg of antiviral compounds, preferably from 1 to 500 mg, more preferably 10 mg, 15 mg, 25 mg, 30 mg, 40 mg, 50 mg, 100 mg, 200 mg, 250 mg, 300 mg, 400 mg, 500 mg, 600 mg, 700 mg or 800 mg.

7. Antiviral pharmaceutical composition, according to claim 4, characterized by the fact that one or more antiviral compounds is present in proportions of about 10 to 100% of the dosage normally administered in a monotherapy regimen.

8. Antiviral pharmaceutical composition, according to claim 4, characterized by the fact that it comprises one or more antiviral compounds selected from remdesivir, AT-527, interferon, interferon-pegylate, ribavirin, acyclovir, cidofovir, docosanol, famciclovir, foscarnet, fomivirisen, ganciclovir, idoxuridine, peciclovir, trifluridine, valacyclovir, vidarabine, amantadines, oseltamivir, zanamivir, peramivir, imiquimod, lamivudine, tenofovir zidovudine, didanosine, stavudina, zalcitabine, indulge, abavavir, neviravir, neviravir, nevavir, nevavir lopinavir, daclastavir, chloroquine, quercetin, vaniprevir, boceprevir, sovaprevir, paritaprevir, telaprevir, favipiravir, palivizumab, ombitasvir, beclabuvir, dasabuvir, pibrantasvir, daclatasvir, remdesivir, other viral inhibitors, RNA inhibitors, other RNA polymerases, other RNA inhibitors, monoclonal or polyclonal antibodies.

9. Antiviral pharmaceutical composition, according to claim 4, characterized by the fact that it further comprises one or more antibiotics selected from amikacin, gentamicin, kanamycin, neomycin, netilmicin, tobramycin, paromomycin, streptomycin, spectinomycin(bs), ansamycins, geldanamycin, herbimycin, rifaximin, carbacephem, loracarbef, carbapenems, ertapenem, doripenem, imipenem/cilastatin, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren, cefoperazone, cefotaxime, cefpodoxime, ceftazidime, ceftibuten, ceftizoxime, moxalactam, ceftriaxone, cefepime, ceftobiprole, teicoplanin, vancomycin, telavancin, dalbavancin, oritavancin, clindamycin, lincomycin, lipopeptide, daptomycin, azithromycin, clarithromycin, erythromycin, roxithromycin, telithromycin, spiramycin, fidaxomicin, monobactams, aztreonam, nitrofurans, furazolidone, nitrofurantoin(bs), oxazolidinones(bs), linezolid, posizolid, radezolid, torezolid, penicillins, amoxicillin, ampicillin, azlocillin, dicloxacillin, flucloxacillin, mezlocillin, methicillin, nafcillin, oxacillin, penicillin g, penicillin v, piperacillin, penicillin g, temocillin, ticarcillin, amoxicillin/clavulanate, ampicillin/sulbactam, piperacillin/tazobactam, ticarcillin/clavulanate, polypeptides, bacitracin, colistin, polymyxin b, quinolones/fluoroquinolones, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, sulfonamides(bs), mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (co-trimoxazole) (tmp-smx), sulfonamidochrysoidine (archaic), tetracyclines(bs), demeclocycline, doxycycline, metacycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol(bs), fosfomycin, fusidic acid, metronidazole, mupirocin, platensimycin, quinupristin/dalfopristin, thiamphenicol, tigecycline(bs), tinidazole, trimethoprim(bs).

10. Antiviral pharmaceutical composition, according to claim 4, characterized by the fact that it additionally comprises a therapeutically effective amount of one or more immunomodulatory compounds.

11. Antiviral pharmaceutical composition, according to claim 10, characterized by the fact that the one or more immunomodulatory compounds are selected from alpha, beta, gamma interferons and pegylated forms of them, glucocorticoids and corticoids.

12. Antiviral pharmaceutical composition, according to claim 4, characterized by the fact that it also includes inactive substances such as dyes, dispersants, sweeteners, emollients, antioxidants, preservatives, pH stabilizers, flavoring, among others, and their mixtures.

13. Antiviral pharmaceutical composition, according to claim 4, characterized by the fact that it is presented in solid or liquid form.

14. Antiviral pharmaceutical composition, according to claim 13, characterized by the fact that the solid form is as a tablet or capsule.

15. Antiviral pharmaceutical composition, according to claim 13, characterized by the fact that the liquid form is as a suspension, solution or syrup.

16. Antiviral pharmaceutical composition, according to claim 4, characterized by the fact that it is for oral or systemic administration.

17. Antiviral pharmaceutical composition, according to claim 16, characterized by the fact that oral administration is by syrup, tablet or capsules.

18. Antiviral pharmaceutical composition, according to claim 16, characterized by the fact that the systemic administration is intravenous, subcutaneous or intramuscular.

19. Combination of compounds characterized by the fact that it comprises (i) compounds as defined in claim 1 and (ii) a synergized amount of raltegravir, dolutegravir and their analogs.

20. Combination of compounds characterized by the fact that it comprises (i) sofosbuvir or tenofovir or their analogs and (ii) raltegranavir and dolutegravir, pibrentasvir, ombitasvir and dacltasvir or their analogs.

21. Combination of compounds, according to claim 19, characterized by the fact that it further comprises one or more antiviral compounds selected from remdesivir, AT-527, interferon, interferon-pegylate, ribavirin, acyclovir, cidofovir, docosanol, famciclovir, foscarnet, fomivirisen, ganciclovir, idoxuridine, peciclovir, trifluridine, valacyclovir, vidarabine, amantadines, oseltamivir, zanamivir, peramivir, imiquimod, lamivudine, tenofovir zidovudine, didanosine, stavudina, zalcitabine, indulge, abavavir, neviravir, neviravir, nevavir, nevavir lopinavir, daclastavir, chloroquine, quercetin, vaniprevir, boceprevir, sovaprevir, paritaprevir, telaprevir, favipiravir, palivizumab, ombitasvir, beclabuvir, dasabuvir, pibrantasvir, daclatasvir, other viral inhibitors, RNA inhibitors, other RNA polymerases, other RNA inhibitors, monoclonal or polyclonal antibodies.

22. Combination of compounds, according to claim 19, characterized by the fact that it further comprises one or more antibiotics selected from amikacin, gentamicin, kanamycin, neomycin, netilmicin, tobramycin, paromomycin, streptomycin, spectinomycin(bs), ansamycins, geldanamycin, herbimycin, rifaximin, carbacephem, loracarbef, carbapenems, ertapenem, doripenem, imipenem/cilastatin, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren, cefoperazone, cefotaxime, cefpodoxime, ceftazidime, ceftibuten, ceftizoxime, moxalactam, ceftriaxone, cefepime, ceftobiprole, teicoplanin, vancomycin, telavancin, dalbavancin, oritavancin, clindamycin, lincomycin, lipopeptide, daptomycin, azithromycin, clarithromycin,erythromycin, roxithromycin, telithromycin, spiramycin, fidaxomicin, monobactams, aztreonam, nitrofurans, furazolidone, nitrofurantoin(bs), oxazolidinones(bs), linezolid, posizolid, radezolid, torezolid, penicillins, amoxicillin, ampicillin, azlocillin, dicloxacillin, flucloxacillin, mezlocillin, methicillin, nafcillin, oxacillin, penicillin g, penicillin v, piperacillin, penicillin g, temocillin, ticarcillin, amoxicillin/clavulanate, ampicillin/sulbactam, piperacillin/tazobactam, ticarcillin/clavulanate, polypeptides, bacitracin, colistin, polymyxin b, quinolones/fluoroquinolones, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, sulfonamides(bs), mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (co-trimoxazole) (tmp-smx), sulfonamidochrysoidine (archaic), tetracyclines(bs), demeclocycline, doxycycline, metacycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol(bs), fosfomycin, fusidic acid, metronidazole, mupirocin, platensimycin, quinupristin/dalfopristin, thiamphenicol, tigecycline(bs), tinidazole, trimethoprim(bs).

23. Combination of compounds, according to claim 19, characterized by the fact that it further a therapeutically effective amount of one or more immunomodulatory compounds.

24. Combination of compounds, according to claim 23, characterized by the fact that one or more immunomodulatory compounds are selected from alpha, beta, gamma interferons and pegylated forms of them, glucocorticoids and corticoids.

25. Use of the antiviral compound or their analogs, as defined in claim 1, characterized by the fact that it is for the manufacture of an antiviral medicine for prophylactic, curative or mitigative treatment of coronavirus, in especial SARS-COV-2, infection and for the treatment of patients with COVID-19 or individuals potentially exposed to coronavirus, in especial SARS-COV-2.

26. Uses, according to claim 25, characterized by the fact that it is for the manufacture of an antiviral drug to inhibit the coronavirus, in especial SARS-CoV-2, RNA synthesis.

27. of the antiviral compound or their analogs as defined in claim 1, characterized by the fact that the drug contains from 1 to 3,000 mg of the antiviral compound as described in claim 1, preferably from 1 to 500 mg, more preferably 10 mg, 15 mg, 25 mg, 30 mg, 40 mg, 50 mg, 100 mg, 200 mg, 250 mg, 300 mg, 400 mg, 500 mg, 600 mg, 700 mg or 800 mg.

28. Uses, according to claim 25, characterized by the fact that it is directed to pregnant women, elderly, individuals with more aggressive manifestations of infections, individuals with more aggressive neurological manifestations of the infection, individuals with mild to severe symptoms of COVID-19, infected with SARS-COV-2, or other coronavirus potentially exposed or at risk of exposure to SARS-COV-2, orally or systemically.

29. Method of treatment prophylactic, curative (therapeutic) or mitigative an individual infected with coronavirus, in especial SARS-COV-2 or potentially exposed to this virus, characterized by the fact that the individual is administered a therapeutically effective amount of one or more antiviral compounds, as defined in claim 1.

30. Method of treatment, according to claim 29, characterized by the fact that the administration of the antiviral compound is oral or systemic.

31. Method of treatment, according to claim 30, characterized by the fact that oral administration is through syrup, capsules, tablets or pills.

32. Method of treatment, according to claim 30, characterized by the fact that the systemic administration is by intravenous, subcutaneous or intramuscular.

33. Method of treatment prophylactic, curative (therapeutic) or mitigative an individual infected with coronavirus, in especial SARS-COV-2 or potentially exposed to this virus, characterized by the fact that the individual is administered a therapeutically effective amount of about 0.01 to about 3,000 mg per kilogram of body weight per day, of the compound as described in claim 1, preferably from 0.03 to 600 mg, more preferably from 0.05 to 400 mg, ideally 25 mg.

34. Method of treatment, according to claim 33, characterized by the fact that the therapeutically effective amount is from 0.05 to 100 mg per kilogram of body weight per day of the compound.

35. Method of treatment, according to claim 33, characterized by the fact that the therapeutically effective amount is from 0.05 to 50 mg per kilogram of body weight per day of the compound.

36. Method of treatment prophylactic, curative (therapeutic) or mitigative an individual infected with coronavirus, in especial SARS-COV-2 or potentially exposed to this virus, Method for the manufacturing of compounds as defined in claim 1, characterized by the fact that it comprises the coupling 6-chloropurines or 6-chloropurine ribosides with appropriate aryl or alkyl amines in the presence of suitable tertiary base, as triethylamine, in alcoholic solvents such as ethanol or isopropanol under reflux conditions, as shown in the following steps:

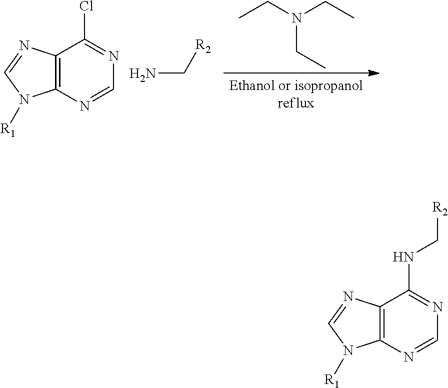

37. Method for the manufacturing of MB-907 compound, characterized by the fact that it comprises the following steps: 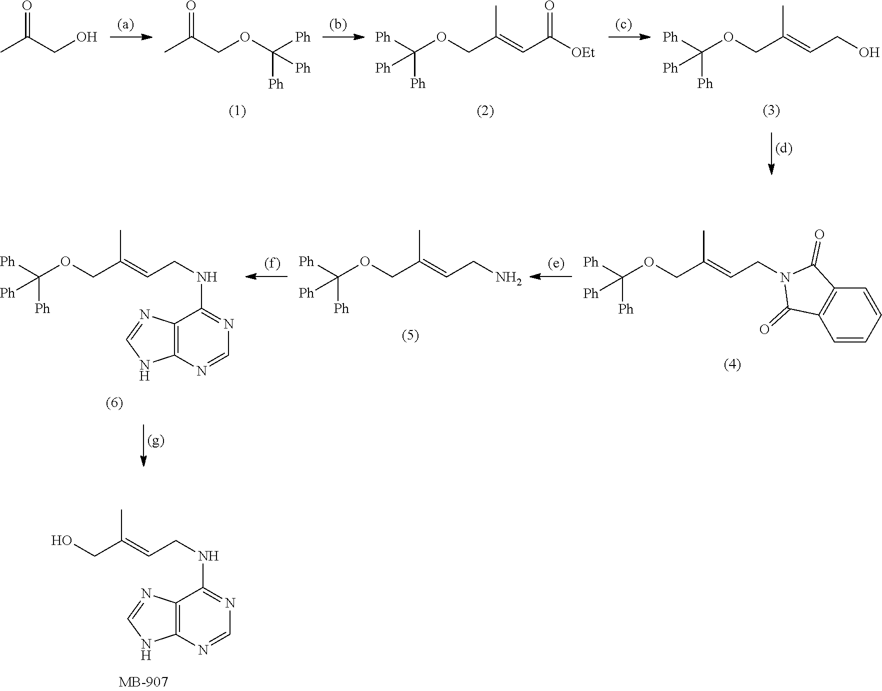
Wherein (a) TrCl, DCM, Py; (b) Ph₃PCHCOOEt, toluene, reflux; (c) LiAlH₄, THF; (d) phthalimide, DIAD, PPh₃, THF; (e) H₄N₂. H₂O, MeOH, reflux; (f) 6-chloropurine, Et₃N, EtOH, reflux; (g) HCl, MeOH, reflux.

38. Method for the manufacturing of MB-711 compound, characterized by the fact that it comprises the following steps: 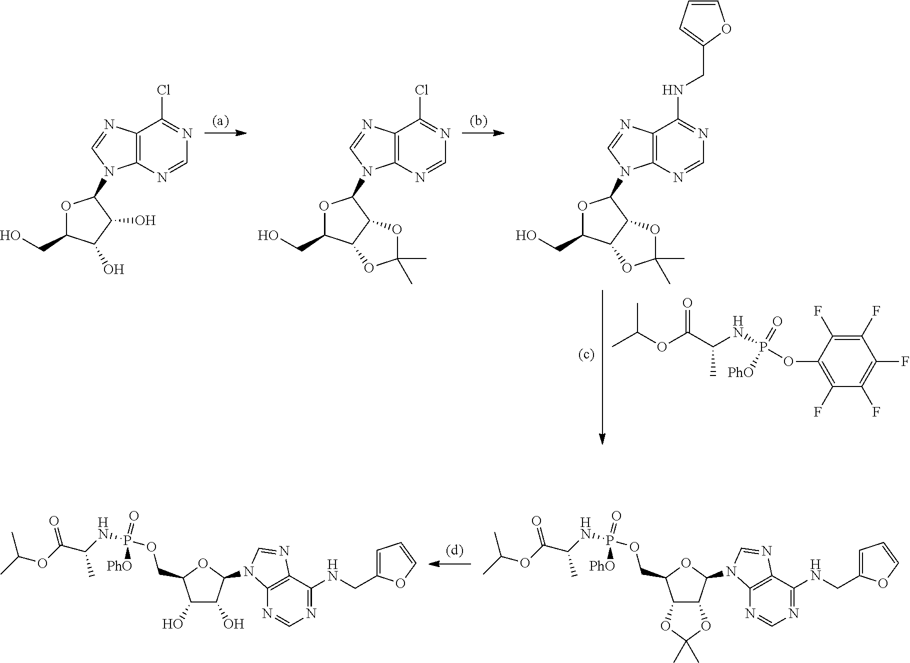
Wherein (a) Me₂(OMe)₂, PTSA, acetone, r.t.; (b) furfuryl amine, Et₃N, EtOH, reflux; (c) t-BuMgCl, THF, 5° to r.t.; (d) CSA(cat), DCM, (CH₂OH)₂, r.t.
